(19) United States
(12) Patent Application Publication
(10) Pub. No.: US 2019/0336969 A1
(43) Pub. Date: Nov. 7, 2019

(54) PARALLELIZED SAMPLE HANDLING

(71) Applicants: CALIFORNIA INSTITUTE OF TECHNOLOGY, Pasadena, CA (US); University of Chicago, Chicago, IL (US)

(72) Inventors: Rustem F. ISMAGILOV, Altadena, CA (US); Liang MA, Pasadena, CA (US); Qichao PAN, Shanghai (CN); Mikhail KARYMOV, Azusa, CA (US); Toan HUYNH, Mountain View, CA (US); George SAWICKI, Chicago, IL (US); Stefano BEGOLO, Marina Del Rey, CA (US); Wenbin DU, Beijing (CN)

(21) Appl. No.: 16/367,036

(22) Filed: Mar. 27, 2019

Related U.S. Application Data
(63) Continuation of application No. 14/785,480, filed on Oct. 19, 2015, now abandoned.
(60) Provisional application No. 61/903,156, filed on Nov. 12, 2013.
(60) Provisional application No. 61/814,090, filed on Apr. 19, 2013.

(51) Int. Cl. B01L 3/00; C12Q 1/686; C12M 3/00; C12Q 1/02; C12M 1/12; B01F 13/00

(52) U.S. Cl. CPC B01L 3/502715; B01L 9/527; C12M 23/44; C12Q 1/025; C12Q 1/02; C12M 23/46; C12M 25/06; B01F 13/0071; B01F 13/0083; B01L 3/502738; B01L 2300/048; B01L 2300/0627; B01L 2300/045; B01L 2200/026; B01L 2200/025; B01L 2200/0642; B01L 2200/0605; C12Q 1/686

(57) ABSTRACT

Provided herein are methods, compositions, and devices for the parallel handling of samples, such as cells or other biological samples. The methods, compositions, and devices are suited for multiple levels of analysis, including genetic and functional assays, of samples.

### CROSS REFERENCE

This application claims the benefit of U.S. Provisional Application No. 61/814,090, filed Apr. 19, 2013, and the benefit of U.S. Provisional Application No. 61/903,156, filed Nov. 12, 2013, which applications are incorporated herein by reference.

### STATEMENT AS TO FEDERALLY SPONSORED RESEARCH

This invention was made with the support of the United States government under Contract number HG005826 by the National Institutes of Health. The United States government has certain rights in the invention.

### BACKGROUND OF THE INVENTION

Cultivation methods that employ miniaturization and compartmentalization, including but not limited to Gel MicroDroplets (GMDs), miniaturized Petri dishes, and microfluidics, can increase throughput, initiate high-density behavior, and eliminate competition from rapidly growing “weed” cells. However, the equipment needed for these methods are often not accessible to most laboratories and operation is complicated, limiting their use for biological assays in the real world.

Microbes play critical roles in environments ranging from soil and oceans to the human gut. Although culture-independent methods such as metagenomics and single-cell genomics have revealed rich information about the composition and function of microbial communities, obtaining pure cultures of these microbes is still fundamental to understanding bacterial genomics and physiology. As bacteria are highly abundant and diverse, it is impractical to cultivate and isolate every strain from the environment.

### SUMMARY OF THE INVENTION

Disclosed herein are methods, devices, systems, and kits for the creation and manipulation of small volumes. In some cases, these are used for control of the micro-environments, including both liquid and gas environments. In some cases, the methods, devices, systems, and kits disclosed herein are used for parallelized sample handling. In some cases, these are used for isolation or cultivation of organisms.

In some embodiments the method comprises: a) providing a substrate comprising a first defined volume, said first defined volume comprising a first sample, said first volume covered by a second substrate; b) providing said second substrate comprising a second defined volume, said second defined volume comprising a second sample, said second defined volume covered by said first substrate; and c) separating said first substrate from said second substrate, whereby said second substrate ceases to cover said first defined volume and said first substrate ceases to cover said second defined volume. In some embodiments, said first sample comprises a gelling agent. In some embodiments, said second sample comprises a gelling agent. In some embodiments, said decoupling is performed on an apparatus, wherein said apparatus maintains the alignment of said first substrate to said second substrate during said decoupling. In some embodiments, said separating occurs by gravity. In some embodiments, said separating occurs by capillary pressure. In some embodiments, said separating occurs by an applied force. In some embodiments, said separating occurs while said first substrate and said second substrate arc submerged in a liquid.

In some embodiments, the method comprises: a) providing a first substrate comprising a first defined volume; b) providing a second substrate comprising second defined volume, said second substrate coupled to said first substrate; c) loading a sample into said first defined volume; d) allowing contents of said first defined volume to enter said second defined volume, thereby producing a combined sample; and e) decoupling said first substrate from said second substrate, wherein a first part of said combined sample remains contained by said first defined volume and a second part of said combined sample remains contained by said second defined volume. In some embodiments the method further comprises, prior to said decoupling, separating said first defined volume from said second defined volume, wherein said first part of said combined sample is contained by said first defined volume and said second part of said combined sample is contained by said second defined volume, wherein said first substrate remains coupled to said second substrate. In some embodiments, said bringing said first defined volume into fluidic contact with said second defined volume is performed on an apparatus. In some embodiments, said decoupling is performed on an apparatus, wherein said apparatus maintains the alignment of said first substrate to said second substrate during said decoupling. In some embodiments, said decoupling is performed on an apparatus, wherein said apparatus comprises a plate for indexing said first defined volume and said second defined volume. In some embodiments, said decoupling occurs by gravity. In some embodiments, said decoupling occurs by capillary pressure. In some embodiments, said decoupling occurs by an applied force. In some embodiments, said decoupling occurs while said first substrate and said second substrate are submerged in a liquid.

In some embodiments, the method comprises: a) providing a first substrate comprising a first defined volume; b) providing a second substrate comprising second defined volume, said second substrate coupled to said first substrate; c) loading a first sample into said first defined volume; d) bringing said first defined volume into fluidic contact with said second defined volume, wherein said first substrate remains coupled to said second substrate; e) mixing contents of said first defined volume with contents of said second defined volume, thereby producing a mixed sample; f) separating said first defined volume from said second defined volume, wherein a first part of said mixed sample is contained by said first defined volume and a second part of said mixed sample is contained by said second defined volume, wherein said first substrate remains coupled to said second substrate; and g) decoupling said first substrate from said second substrate, wherein said first part of said mixed sample remains contained by said first defined volume and said second part of said mixed sample remains contained by said second defined volume. In some embodiments, said mixed sample further comprises a gelling agent. In some embodiments, said mixing comprises diffusion. In some embodiments, said mixing comprises sonication. In some embodiments, said bringing said first defined volume into fluidic contact with said second defined volume is performed on an apparatus. In some embodiments, said decoupling is performed on an apparatus, wherein said apparatus maintains the alignment of said first substrate to said second substrate during said decoupling. In some embodiments, said decoupling is performed on an apparatus, wherein said apparatus maintains the alignment of said first substrate to said second substrate during said decoupling. In some embodiments, said decoupling occurs by gravity. In some embodiments, said decoupling occurs by capillary pressure. In some embodiments, said decoupling occurs by an applied force. In some embodiments, said decoupling occurs while said first substrate and said second substrate are submerged in a liquid.

In some embodiments, the device comprises: a) a first substrate comprising a first defined volume and a channel; b) a second substrate comprising second defined volume, said second substrate coupled to said first substrate; and c) an immiscible fluid layer disposed between said first substrate and said second substrate. In some embodiments, gas contained in said channel enters said first defined volume and said second defined volume by diffusion through said immiscible fluid layer. In some embodiments, gas contained in said channel enters said first defined volume and said second defined volume by diffusion through said first substrate and said second substrate. In some embodiments, said second substrate further comprises a channel. In some embodiments, the device further comprises posts located on said first substrate, positioned between said first substrate and said second substrate. In some embodiments, the device further comprises posts located on said second substrate, positioned between said first substrate and said second substrate. In some embodiments, the device further comprises means for temperature control of said first substrate and said second substrate.

In some embodiments, the method, comprises: a) dispersing a sample among a plurality of defined volumes; b) splitting said plurality of defined volumes, essentially simultaneously, into a plurality of matched pairs of daughter volumes comprising a plurality of first daughter volumes and a plurality of matched second daughter volumes, wherein said splitting is performed without the application of a pumping force to said defined volumes; c) conducting at least one analysis on said plurality of said first daughter volumes; and d) selecting a subset of said plurality of matched second daughter volumes based on said analysis. In some embodiments, said analysis comprises a genetic assay. In some embodiments, said analysis comprises a functional assay. In some embodiments, said sample comprises cells. In some embodiments, said sample comprises bacterial cells. In some embodiments, said sample comprises mammalian cells. In some embodiments, said sample comprises viruses. In some embodiments, said sample comprises nucleic acids. In some embodiments, said sample comprises multiple species of cells. In some embodiments, said sample comprises antibiotics. In some embodiments, said sample comprises chemotherapy agents. In some embodiments, said sample comprises growth media. In some embodiments, said sample comprises growth factors. In some embodiments, said sample comprises inhibitors.

In some embodiments, the method comprises: a) loading fluid into a container; b) bringing an opening in said container into contact with a sample fluid volume; c) allowing said sample fluid volume to merge by surface tension with said fluid in said container, producing a merged liquid volume; and d) expelling said merged liquid volume from said container.

In some embodiments, the device comprises an assembly capable of performing, in an automated fashion the following protocol: a) loading fluid into a container; b) bringing an opening in said container into contact with a sample fluid volume; c) allowing said sample fluid volume to merge by surface tension with said fluid in said container, producing a merged liquid volume; and d) expelling said merged liquid volume from said container. In some embodiments, said protocol is performed for a plurality of sample fluid volumes in parallel. In some embodiments, said protocol is performed for a plurality of sample fluid volumes in series. In some embodiments, the volume of said sample fluid volume is less than or equal to 5 microliters. In some embodiments, the volume of said sample fluid volume is less than 100 nanoliters.

In some embodiments, the method comprises: a) providing a plurality of defined volumes, each of said plurality of defined volumes comprising one of a plurality of samples; b) transferring said plurality of samples from said plurality of defined volumes to a shared container, thereby creating a pooled sample; and c) conducting an analysis on said pooled sample. In some embodiments, said plurality of defined volumes are defined by one substrate. In some embodiments, said plurality of defined volumes are defined by multiple substrates. In some embodiments, said analysis comprises a genetic assay. In some embodiments, said analysis comprises a functional assay.

In some embodiments, the method comprises: a) providing a plurality of defined volumes, each of said plurality of defined volumes comprising one of a plurality of samples; b) subjecting said plurality of samples to a set of conditions; c) conducting a process on said plurality of samples; d) transferring said plurality of samples from said plurality of defined volumes to a shared container, thereby creating a pooled sample; e) conducting an analysis on said pooled sample; and f) determining from said analysis the extent to which said set of conditions enabled or did not enable said process. In some embodiments, said set of conditions comprises the presence of an antibiotic. In some embodiments, said set of conditions comprises the presence of a chemotherapy agent. In some embodiments, said set of conditions comprises a given temperature. In some embodiments, said set of conditions comprises a given atmospheric composition. In some embodiments, said set of conditions comprises the presence of a given organism. In some embodiments, said set of conditions comprises the presence of a growth factor. In some embodiments, said set of conditions comprises the presence of an inhibitor. In some embodiments, said set of conditions comprises the absence of a nutrient. In some embodiments, said process comprises cell growth. In some embodiments, said process comprises nucleic acid amplification. In some embodiments, said analysis comprises a genetic assay. In some embodiments, said analysis comprises a functional assay. In some embodiments, said analysis comprises detection of organisms associated with mastitis. In some embodiments, said analysis comprises detection of organisms associated with IBD. In some embodiments, said analysis comprises detection of organisms associated with sulfate-reduction. In some embodiments, said analysis comprises detection of organisms useful as probiotics.

### INCORPORATION BY REFERENCE

All publications, patents, and patent applications mentioned in this specification are herein incorporated by reference in their entireties for any and all purposes to the same extent as if each individual publication, patent, or patent application was specifically and individually indicated to be incorporated by reference, including: U.S. Application 61/516,628, U.S. Application 61/518,601, U.S. application Ser. No. 13/257,811, U.S. application Ser. No. 12/670,739, international application PCT/US2010/028361, U.S. Application 61/262,375, U.S. Application 61/162,922, U.S. Application 61/340,872, U.S. application Ser. No. 13/440,371, U.S. application Ser. No. 13/467,482, U.S. application Ser. No. 13/868,028, U.S. application Ser. No. 13/868,009, and international application PCT/US13/63594.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a holder with three alignment pins and two glass spacers.

FIG. 2A shows a side-view schematic of splitting with a holder.

FIG. 2B shows a side-view schematic and a top-view photograph of a device before splitting.

FIG. 2C shows a side-view schematic and a top-view photograph of a device after splitting.

FIG. 2D shows a photograph of a device after splitting.

FIG. 3A shows defined volumes comprising liquid sample with a pipette tip.

FIG. 3B shows a liquid sample merging with liquid in a pipette tip.

FIG. 3C shows a defined volume with liquid and sample removed.

FIG. 4A shows loading of aqueous solution on a device for chip wash.

FIG. 4B shows compartmentalization on a device for chip wash.

FIG. 4C shows removal of residual aqueous solution on a device for chip wash.

FIG. 4D shows actuation of a device to enable chip wash of partitioned samples.

FIG. 5A shows a schematic of cultivation of single E. coli expressing GFP and DsRed genes, a schematic and a photograph of PCR identification of E. coli, and a schematic and a photograph of fluorescence microscopy identification of E. coli.

FIG. 5B shows a montage of microscopy and PCR results.

FIG. 5C shows a visualization of microscopy and PCR identification of E. coli.

FIG. 6A shows a SlipChip used for digital PCR.

FIG. 6B shows a SlipChip with gas supply channels.

FIG. 7 shows illustrations and photographs of anaerobic cultivation of B. theta on a SlipChip device.

FIG. 8A shows a photograph of a device with vertical wells loaded with bacteria culture.

FIG. 8B shows a photograph of a device with vertical wells loaded with bacteria culture.

FIG. 8C shows 900 nm height nanoposts.

FIG. 8D shows growth of E. coli in a SlipChip device with no nanoposts.

FIG. 8E shows growth of E. coli in a SlipChip device with 400 nm height nanoposts.

FIG. 8F shows growth of E. coli in a SlipChip device with 900 nm height nanoposts.

FIG. 9A shows a schematic of a device in a controlled atmosphere bottle.

FIG. 9B shows devices in three bottles with differing oxygen concentrations.

FIG. 9C shows growth of B. theta and E. coli under differing oxygen conditions.

FIG. 10A shows a line scan of isolated compartments.

FIG. 10B shows a line scan of diffusively connected compartments.

FIG. 10C shows a cross-section schematic of isolated compartments.

FIG. 10D shows a cross-section schematic of diffusively connected compartments.

FIG. 11 shows growth of A. caccae alone and with B. theta in chemical communication.

FIG. 12A shows an empty SlipChip with overlapping wells and channels.

FIG. 12B shows sample loaded into a SlipChip.

FIG. 12C shows a SlipChip slipped to overlap the wells in the two plates.

FIG. 12D shows a SlipChip with channels flushed with air.

FIG. 12E shows a SlipChip with channels flushed with oil.

FIG. 12F shows a SlipChip slipped to separate overlapping wells.

FIG. 13 shows fluid volumes without agarose after SlipChip splitting.

FIG. 14A shows cultivation under stochastic confinement of a slow-growing strain from a microbial community.

FIG. 14B shows distribution of cells on two sides of a SlipChip after slipping and separating.

FIG. 15A shows the top layer of a dilution device, with 1× volume wells.

FIG. 15B shows the bottom layer of a dilution device, with 9× volume wells.

FIG. 15C shows the top and bottom layers of a dilution device assembled.

FIG. 15D shows a dilution device filled with sample.

FIG. 15E shows the top plate of a dilution device slipped to separate wells from ducts.

FIG. 15F shows the first step of dilution.

FIG. 15G shows 100,000-fold dilution after five steps of dilution.

FIG. 16 shows three steps of two-fold serial dilution for eight-fold total dilution.

FIG. 17A shows steps for fabricating hydrophilic wells.

FIG. 17B shows aqueous solution in hydrophilic wells.

FIG. 18 shows the results of on-device dilution.

FIG. 19 shows fluid volumes comprising 1% agarose on half of a SlipChip device.

FIG. 20A shows a schematic of sequencing for targeted cultivation and isolation of a microbial target organism on a device.

FIG. 20B shows a schematic of chip wash for targeted cultivation and isolation of a microbial target organism on a device.

FIG. 20C shows a schematic of splitting and PCR for targeted cultivation and isolation of a microbial target organism on a device.

FIG. 21 shows results of chip wash qPCR with E. coli.

FIG. 22A shows growth of C. scindens on a SlipChip device.

FIG. 22B shows growth E. faecalis on a SlipChip device.

FIG. 22C shows growth of C. scindens on an agar plate.

FIG. 22D shows growth E. faecalis on an agar plate.

FIG. 22E shows a graph comparing genomic DNA of C. scindens and E. faecalis collected from a non-growth negative control, a chip wash method, and a plate wash method.

FIG. 23 shows a schematic of sample collection and confinement.

FIG. 24 shows qPCR results with target specific primers and universal primers.

FIG. 25A shows positive and negative on-chip colony PCR on a split SlipChip.

FIG. 25B shows a scaled up colony of “Candidatus Caecococcus microfluidicus.”

FIG. 25C shows a micrograph of a single colony of “Candidatus Caecococcus microfluidicus.”

FIG. 25D shows a TEM image of “Candidatus Caecococcus microfluidicus.”

FIG. 26 shows optical microscopy of OTU158.

FIG. 27A shows the phylogenetic affiliation of “Candidatus Caecococcus microfluidicus.”

FIG. 27B shows validation of “Candidatus Caecococcus microfluidicus” culture by FISH.

FIG. 28 shows a schematic of cultivation and isolation of a target organism.

### DETAILED DESCRIPTION OF THE INVENTION

I. Overview

This disclosure provides methods and compositions for the parallel handling of samples. The parallel sample handling can be conducted by a variety of means. For example, the sample handling can occur on a SlipChip device. The SlipChip device can comprise two facing plates each comprising a series of wells, chambers, or other defined volumes. Sample handling including loading, mixing, reactions, separations, dilutions, presentation of results, and other steps can occur by actuation of the SlipChip plates relative to each other. Parallelization of samples can occur by splitting the two plates, producing spatially ordered matching sets of samples on each of the two plates. Further analysis of the parallel sample sets can be conducted on one or both sets of samples, including analyses that are destructive or consumptive of the analytes.

This disclosure describes a method for a) stochastic confinement and cultivation from a single cell; b) creation of replica of micro-colonies; c) targeted isolation of microbes using gene-based assays; and d) targeted isolation of microbes using function-based assays. The method can, for example, be performed on a SlipChip device. SlipChip is a microfluidic device that can manipulate fluid volumes, e.g. picoliter- to nanoliter-sized fluid volumes, which in some cases may not require complex equipment. In some cases, single cells are stochastically confined (for example, in a 1,000-compartment SlipChip) and incubated to allow growth of colonies. In addition to confinement, the microenvironment around the microbes can be tuned by controlling the strength and direction of interaction between microbes, if desired. In some cases, the cultivar from the microdevice can be pooled together and collected into one tube to run high throughput multiplexed assays. In some cases, when the two plates of the device are separated, each compartmentalized fluid volume splits into two, creating copies of each individual colony on each of the opposing plates. For gene-based assays, a reaction, such as PCR, can be performed on the first plate to identify the compartments containing the colonies with the gene of interest. Corresponding colonies can then be retrieved from the second plate for other applications, including but not limited to scale-up culture of isolates of interest.

II. SlipChip

The methods, compositions, devices, and kits disclosed herein may be used with SlipChip devices. SlipChip devices are described, for example, in international patent application PCT/US2010/028361, “Slip Chip Device and Methods,” filed on Mar. 23, 2010; U.S. application Ser. No. 13/257,811, “Slip Chip Device and Methods,” filed on Sep. 20, 2011; U.S. Application 61/262,375, “Slip Chip Device and Methods,” filed on Nov. 18, 2009; U.S. Application 61/162,922, “Sip Chip Device and Methods,” filed on Mar. 24, 2009; U.S. Application 61/340,872, “Slip Chip Device and Methods,” filed on Mar. 22, 2010; U.S. Application 61/516,628, “Digital Isothermal Quantification of Nucleic Acids Via Simultaneous Chemical Initiation of Recombinase Polymerase Amplification (RPA) Reactions on Slip Chip,” filed on Apr. 5, 2011; and United States Application 61/518,601, “Quantification of Nucleic Acids With Large Dynamic Range Using Multivolume Digital Reverse Transcription PCR (RT-PCR) On A Rotational Slip Chip Tested With Viral Load,” filed on May 9, 2011.

In brief, SlipChip devices are micro-fluidic devices which can comprise plates coupled to each other. Each plate can comprise a plurality of wells, compartments, or other defined volumes. The plates can move relative to each other. Motion of the plates can result in various on-chip operations, such as bringing defined volumes on one plate into or out of fluid contact with wells on another plate, or bringing defined volumes into or out of fluid contact with an inlet or outlet channel.

SlipChip devices can be fabricated from a variety of materials, such as glass, silicon, polymers (e.g. PMMA or PDMS), or metal. SlipChip devices can be fabricated by a variety of methods, such as photolithography, soft lithography, hot embossing, laser ablation, wet etching, plasma etching (e.g. RIE or DRIE), or micromolding.

SlipChip devices can comprise a variety of lubricating phases between the components, such as perfluorinated compounds, mineral oil, or other oils.

SlipChip devices may be prepared with a variety of surface treatments, such as silanization, oxygen plasma activation, polymer coatings (e.g. PDMS or parylene), affinity agents, metals, electrodes, dielectrics, proteins, hydrophilic coatings and treatments, or hydrophobic coatings and treatments.

SlipChip devices may comprise a variety of additional functional components, including heaters, Peltier devices, piezoelectric actuators, pumps, light sources, optical sensors, CCDs, magnets, and other components.

III. Samples

The samples handled in this disclosure can comprise cells. The samples can comprise a single type of cells, organisms, or viruses. The samples can comprise multiple types of cells, organisms, or viruses. The samples can comprise multiple species of microbe or microbial consortia. The samples can comprise bacterial cells. The samples can comprise fungal cells. The samples can comprise mammalian cells. The samples can comprise insect cells. The samples can comprise tumor cells. The samples can comprise viruses. The samples can comprise algae. The samples can comprise archaea. The samples can comprise E. coli, F. faecalis, A. caccae, B. vulgatus, B. theta, or other species. The samples can comprise human cells, such as HeLa, NCI60, DU145, HUVEC, Jurkat, Lncap, MCF-7, MDA-MB-438, PC3, T47D, THP-1, U87, SHSY5Y, or Saos-2. The samples can comprise non-human animal cells, such as Vero, GH3, PC12, MC3T3, Zebrafish ZF4, Zebrafish AB9, MDCK, or Xenopus A6. The samples can comprise stem cells. The samples can comprise previously undiscovered or uncharacterized species. The samples can comprise plant cells, such as tobacco BY-2. The samples can comprise organisms or communities of organisms selected based on a marker or function, such as a gene, a set of genes, a genetic marker or set of genetic markers, phenotypic characteristics, or activities of interest such as cellulose degradation, lignin degradation, fermentation, infection, sulfate reduction, or other activities. The samples can comprise isolates from any of the foregoing cells or organisms.

The samples can comprise reagents, such as enzymes, nucleotides, oligonucleotides, primers, labels, probes, particles, lysis agents, sample preparation reagents for sequencing or next-generation sequencing, inducers (e.g. IPTG), repressors (e.g. LacI), signaling molecules (e.g. hormones), or other reagents. The samples can comprise reaction product, such as PCR or digital PCR product. The samples can comprise other reaction products. The samples can comprise nucleic acids, such as DNA, RNA, PNA, plasmids, regulatory or non-coding RNA, or other nucleic acids.

The samples can comprise media, such as cooked meat medium, AM2 medium, LB medium, LB Miller medium, LB Lennox medium, SOB medium, SOC medium, 2× YT medium, TB medium, SB medium, or other commercially available media.

The samples can comprise buffer, such as Bicine, Tris, Tricine, TAPSO, HEPES, TES, TAPS, PBS, MOPS, PIPES, Cacodylate, SSC, MES, succinic acid, Good's buffers, or other commercially available buffers.

The samples can comprise gas, such as O₂, CO₂, CO, N₂, NO, NO₂, H₂O, or air.

The samples can comprise growth factors or cytokines such as adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β, Tumor necrosis factor-alpha (TNF-α), Vascular endothelial growth factor (VEGF), Wnt Signaling Pathway, placental growth factor (PGF), Foetal Bovine Somatotrophin (FBS), IL-1, IL-2, IL-3, IL-4, IL-5, IL-6, IL-7, or other growth factors.

The samples can comprise antibiotics, such as ampicillin, carbenicillin, chloramphenicol, D-cycloserine, gentamycin, hygromycin B, kanamicin, kasugamycin, nalidixic acid, neomycin, rifampicin, spectinomycin, streptomicin, tetracycline, or other antibiotics.

The samples can comprise antibodies, antibody fragments, or aptamers. The samples can comprise binding agents capable of binding to specific targets. The samples can comprise fluorophores.

The samples can comprise fluid volumes. A fluid volume can be a droplet. A fluid volume can be contained, completely or partially, in a well, compartment, or other structure. A fluid volume can be on a substrate. A fluid volume can be in an emulsion. A fluid volume can be defined by surface tension. A fluid volume can be defined by one or more solid substrates. A fluid volume can be defined by one or more immiscible liquids. A fluid volume can be defined by a gas phase. A fluid volume can be defined by a combination of solid, liquid, and/or gas phases.

IV. Parallel Handling of Samples

Many assays provide valuable information but may destroy or consume their analytes in the process. For such assays, the present disclosure provides parallel sample handling in defined volumes, producing two matched sets of samples from one original set of samples. In some cases, one set can be used for an analysis that destroys or consumes its analytes and another set can be preserved for reference, amplification or growth, or further study. In some other cases, one set can be used for a first analysis that destroys or consumes its analytes and another set can be used for a second analysis that destroys or consumes its analytes. For example, genetic assays such as PCR or FISH often require cells to be lysed to analyze their genetic material, although it is desirable to obtain living micro-organisms for microbial isolation. In some cases, multiple copies of an analyte are provided to each defined volume in the original set of samples, and each of the two matched sets of samples produced contain fewer analytes than the original volume. In some cases, at least one copy of an analyte is provided to each defined volume in the original set of samples, and amplification of the analytes is performed prior to production of the two matched sets of samples. The amplification can be cell growth. The amplification can be by nucleic acid amplification.

The parallel sample handling can be performed by a SlipChip device. The SlipChip device can comprise opposing plates, each with a given number of defined volumes. The defined volumes can be compartments or wells. The wells can be loaded with samples and subsequently the plates can be slipped relative to each other to bring compartments or wells on one plate in contact with the corresponding compartments or wells on another plate. For example, a suspension of cells can be loaded into the wells on both plates, and the wells can then be brought together by slipping and form combined wells. Alternatively, a suspension of cells can be loaded onto the wells on one plate and a different solution, such as growth media, can be loaded onto the wells on another plate, and the wells can then be brought together by slipping to form combined wells. These combined wells can form the original set of samples. Formation of matched sets of samples can then be formed by slipping the plates to separate the sets of wells.

The parallel sample handling can be performed by a droplet microfluidic device. Droplets can be aqueous droplets partitioned by an immiscible oil phase. Droplets can be oil droplets partitioned by an immiscible aqueous phase. A population of droplets comprising an original set of samples can undergo sequential droplet splitting, producing two matched sets of samples in the daughter droplets.

The parallel sample handling can be performed by other defined volume systems. In some cases, the original set of samples comprises aqueous samples in a microwell plate. The two matched sets of samples can be produced by transferring a fraction of the original sample volume from the original set of samples into a second microwell plate. Transferring can be conducted by pipetting, for example.

V. Splitting SlipChip

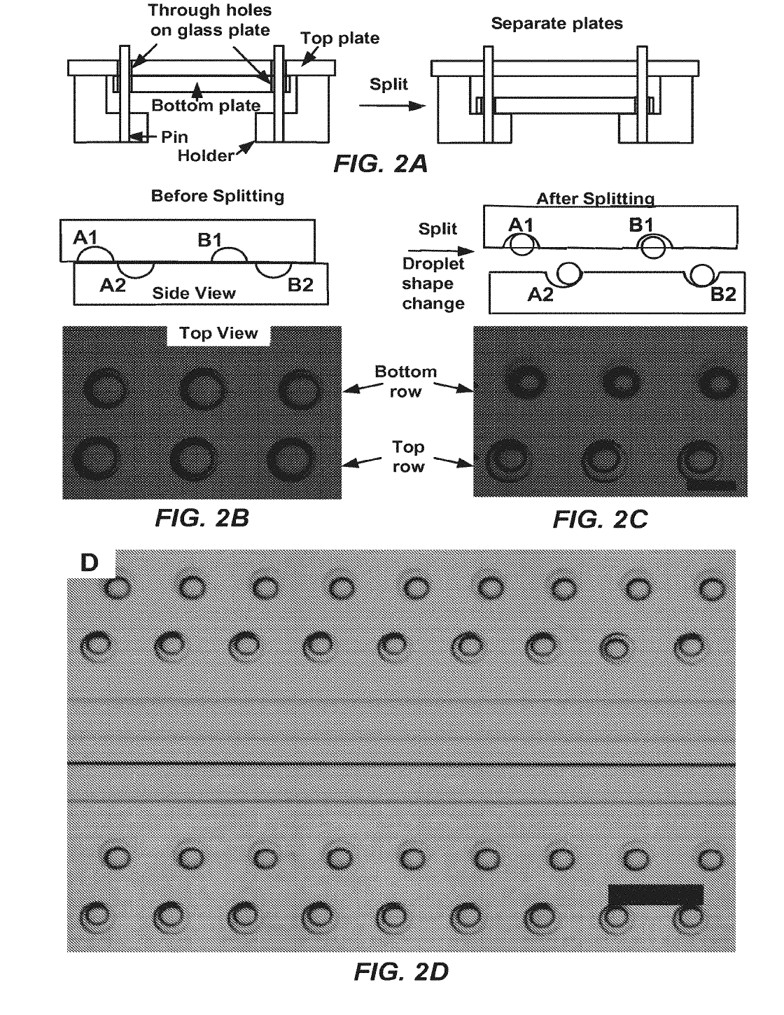

The parallel sample handling can be performed by a SlipChip device, which can comprise two opposing plates, each with a given number of defined volumes. As described above, formation of two matched sets of samples from the original set of samples can be formed by slipping the two plates to separate the two sets of defined volumes (e.g. FIG. 2B, FIG. 2C). These two matched sets of samples can be further separated by separating the two opposing plates of the SlipChip device, enabling access to each sample for further analysis while preserving the paired relationship between members of the two matched sets of samples, based on their physical location in the array of defined volumes.

The plates can be decoupled without the requirement to first separate the first defined volume from the second defined volume. In such cases, decoupling of the two substrates provides separation of the two volumes. The cohesion force within the sample can be smaller than the adhesion of between the sample and the substrate. For example, all or a part of the substrate can be hydrophilic and the sample can be aqueous. For example, see FIG. 17 where a substrate with hydrophilic surface inside the wells and a hydrophobic surface outside of the wells was generated FIG. 17A) and an aqueous sample was loaded (FIG. 17B). In another example, hydrophilic wells surrounded by hydrophobic surface were used for protein assays (See Weishan Liu, Delai Chen, Wenbin Du, Kevin P. Nichols, and Rustem F. Ismagilov, “SlipChip for Immunoassays in Nanoliter Volumes,” Analytical Chemistry 2010 82:3276-3282). In some cases, the substance inside the volume can be adhesive to the substrate. For example, the substance can be mammalian cells that are adherent to surfaces.

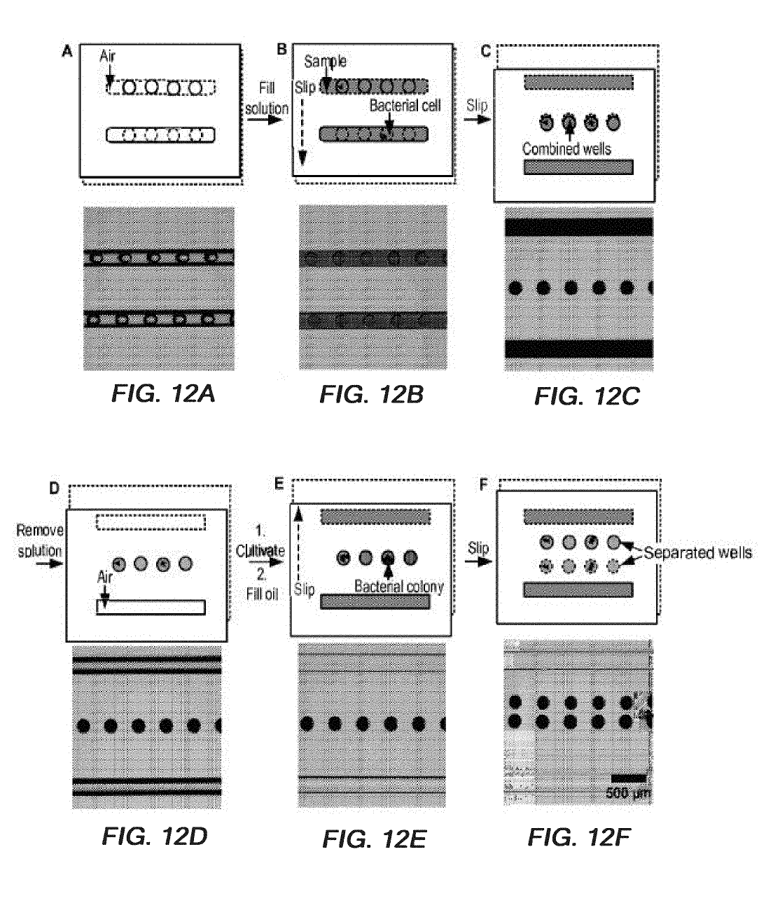

In some cases, one substrate comprises a volume, another substrate comprises another volume, and those volumes are not fluidically connected and may have the same or different contents. The two substrates can be decoupled. Decoupling can provide access to the volumes. This can be prepared by using a SlipChip, for example. Such volumes can be prepared by slipping apart a combined volume containing a mixture. For one example, see FIG. 12.

Volumes, including fluidically connected or fluidically disconnected volumes, can be similar or different in volume (See Feng Shen, Bing Sun, Jason E. Kreutz, Elena K. Davydova, Wenbin Du, Poluru L. Reddy, Loren J. Joseph, and Rustem F. Ismagilov, “Multiplexed Quantification of Nucleic Acids with Large Dynamic Range Using Multivolume Digital RT-PCR on a Rotational SlipChip Tested with HIV and Hepatitis C Viral Load,” JACS 2011 133: 17705-17712 and Jason E. Kreutz, Todd Munson, Toan Huynh, Feng Shen, Wenbin Du, and Rustem F. Ismagilov, “Theoretical Design and Analysis of Multivolume Digital Assays with Wide Dynamic Range Validated Experimentally with Microfluidic Digital PCR,” Analytical Chemistry 2011 83: 8158-8168). Volumes can be configured for nucleic acid amplification reactions (See Feng Shen, Elena K. Davydova, Wenbin Du, Jason E. Kreutz, Olaf Piepenburg, and Rustem F. Ismagilov, “Digital Isothermal Quantification of Nucleic Acids via Simultaneous Chemical Initiation of Recombinase Polymerase Amplification Reactions on SlipChip,” Analytical Chemistry 2011 83:3533-3540; Feng Shen, Bing Sun, Jason E. Kreutz, Elena K. Davydova, Wenbin Du, Poluru L. Reddy, Loren J. Joseph, and Rustem F. Ismagilov, “Multiplexed Quantification of Nucleic Acids with Large Dynamic Range Using Multivolume Digital RT-PCR on a Rotational SlipChip Tested with HIV and Hepatitis C Viral Load,” JACS 2011 133: 17705-17712; Jason E. Kreutz, Todd Munson, Toan Huynh, Feng Shen, Wenbin Du, and Rustem F. Ismagilov, “Theoretical Design and Analysis of Multivolume Digital Assays with Wide Dynamic Range Validated Experimentally with Microfluidic Digital PCR,” Analytical Chemistry 2011 83: 8158-8168; and Feng Shen, Wenbin Du, Jason E. Kreutz, Alice Fok, and Rustem F. Ismagilov, “Digital PCR on a SlipChip,” Lab Chip 2010 10: 2666-2672). Volumes can be configured for growth of organisms, including microorganisms, cells, etc. as described herein. Volumes can be configured for crystallization, including but not limited to protein crystallization (See Liang Li and Rustem F. Ismagilov, “Protein Crystallization Using Microfluidic Technologies Based on Valves, Droplets, and SlipChip, Annu. Rev. Biophys 2010 39: 139-158 and Liang Li, Wenbin Du, and Rustem F. Ismagilov, “Multiparameter Screening on SlipChip Used for Nanoliter Protein Crystallization Combining Free Interface Diffusion and Microbatch Methods,” JACS 2010 132: 112-119). Volumes can be configured for protein assays (Weishan Liu, Delai Chen, Wenbin Du, Kevin P. Nichols, and Rustem F. Ismagilov, “SlipChip for Immunoassays in Nanoliter Volumes,” Analytical Chemistry 2010 82:3276-3282). Single cells and molecules can be amplified and retrieved. Surfaces can be hydrophilic or hydrophobic (Weishan Liu, Delai Chen, Wenbin Du, Kevin P. Nichols, and Rustem F. Ismagilov, “SlipChip for Immunoassays in Nanoliter Volumes,” Analytical Chemistry 2010 82:3276-3282).

Separation of the two SlipChip plates can be performed underneath a fluid. The fluid can comprise immiscible oil, such as tetradecane oil, perfluorinated oil, or any other suitable immiscible liquid. Evaporation can, in some cases, be reduced or not occur, on the SlipChip.

The force used to separate the two SlipChip plates can be gravity. The force used to separate the two SlipChip plates can be capillary force. The force used to separate the two SlipChip plates can be hydrodynamic. The force used to separate the two SlipChip plates can be applied via an implement, such as tweezers, a razor blade, or a finger.

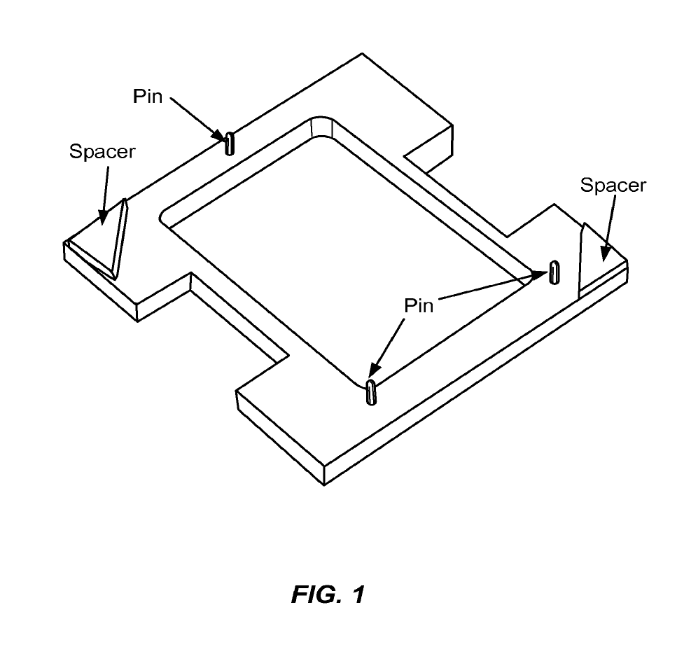

The SlipChip plates can be held in position relative to each other during separation to ensure separation of sample volumes or droplets is maintained. SlipChip plates can be held in position manually. SlipChip plates can be held in position with clamps. SlipChip plates can be held in position with a holder (e.g. FIG. 1, FIG. 2A).

Splitting of SlipChip plates can be further enabled by increasing the viscosity of the samples to prevent sample volumes or droplets from becoming dislodged during splitting. The viscosity of the samples can be increased by the addition of polymers. The viscosity of the samples can be increased by the addition of gels. The viscosity of the samples can be increased by the addition of high viscosity liquids. The viscosity of the samples can be increased by temperature change. In some cases, the viscosity of the samples can be increased by the addition of glycerol. In some cases, the viscosity of the samples is increased by the addition of agarose. The agarose can be ultra-low gelling temperature agarose. The concentration of the agarose can be at least 0.1%. The concentration of the agarose can be at least 0.2%. The concentration of the agarose can be at least 0.3%. The concentration of the agarose can be at least 0.4%. The concentration of the agarose can be at least 0.5%. The concentration of the agarose can be at least 0.6%. The concentration of the agarose can be at least 0.7%. The concentration of the agarose can be at least 0.8%. The concentration of the agarose can be at least 0.9%. The concentration of the agarose can be at least 1.0%. The concentration of the agarose can be at least 1.2%. The concentration of the agarose can be at least 1.4%. The concentration of the agarose can be at least 1.6%. The concentration of the agarose can be at least 1.8%. The concentration of the agarose can be at least 2.0%. In some cases, agarose is added to the sample at a concentration between about 0.3% and about 2.0%. The temperature of the samples can be lowered to gellify the agarose. SlipChip plates can be split subsequent to gelling of the samples (e.g. FIG. 2D).

In some cases, the defined volumes on one SlipChip plate are loaded with a solution comprising organisms and the defined volumes on the other SlipChip plate are loaded with a solution comprising agarose. The organisms can be cultured in the absence of agarose, and after organism growth the two sets of volumes can be combined. These combined volumes can form the original set of samples. Formation of two matched sets of samples can then be formed by slipping the two plates to separate the two sets of volumes.

VI. Retrieval of Samples

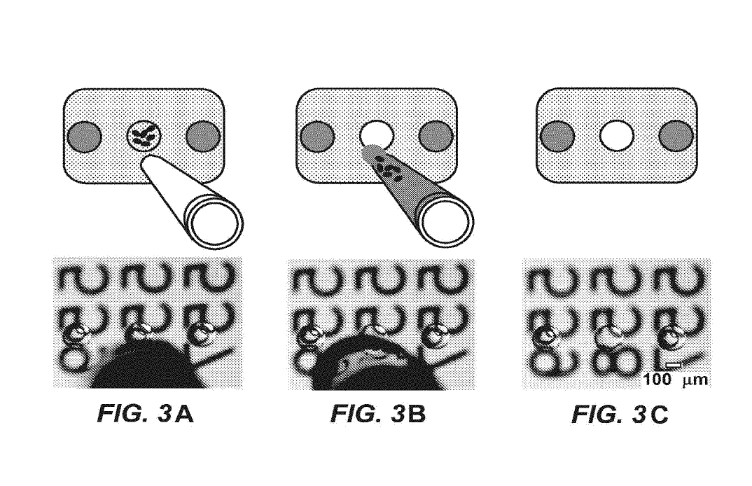

Subsequent to parallelizing, the samples can be retrieved for further analysis. Samples can be retrieved by surface-to-surface transfer. Samples can be retrieved by pipetting. Pipetting can be conducted by first pipetting a volume of buffer solution into defined volume and allowing the buffer to mix with the sample volume. The entire volume of buffer and sample can then be pipetted up and transferred. The spacing between wells on the SlipChip can be larger than the outer radius of the pipette tip to prevent cross-contamination between wells. Alternatively, pipetting can be conducted by bringing a pipet tip filled with solution, such as buffer, into contact with the sample volume, allowing the sample volume to merge with the liquid in the pipet tip (e.g. FIG. 3).

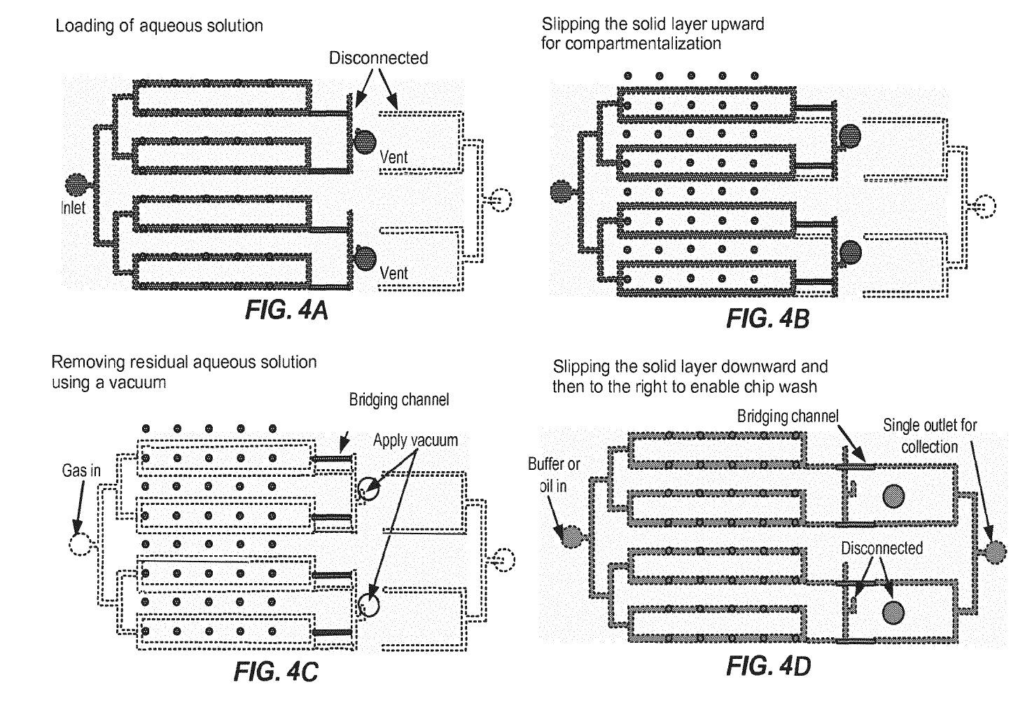

Samples can be retrieved by a chip wash method. A chip wash method can be used to monitor a reaction or other process on a device under various conditions. Samples contained in defined volumes can be washed out of a device, such as a SlipChip device, and aggregated. Analyses then can be performed on the aggregated samples. Analyses can determine the extent to which the examined conditions enable or disable the reaction or other process. Analysis can, for example, determine the minimum condition for enabling the process, the optimum condition for enabling the process, intermediate conditions for enabling the process, or minimum conditions for disabling the process. For example, DNA can be partitioned into defined volumes on a device or chip, amplified, washed into a combined volume, and then the combined product can then be analyzed to determine if that amplification condition supports the amplification of the desired target. In another example, cells can be partitioned into defined volumes on a device or chip, cultivated, washed from the device into a combined volume, and then DNA from pooled cells can be analyzed by sequencing, target-specific primers, or both, in order to determine whether the cultivation conditions which were used supported the growth of target microorganisms. This chip wash method can be repeated sequentially or in parallel until conditions are identified for the target. For example, a SlipChip based microfluidic device can designed for this chip wash method to be capable of performing up to 3,200 microbial cultivation experiments, each on a scale of ˜6 nL. Such a device can enable three capabilities: stochastic confinement of single cells from samples, microbial cultivation, and collection of cultivated cells. Such a device can permit collect the chip wash solution with a single outlet. The design is illustrated in FIG. 4. This embodiment of the design features can use bridging channels to direct the flow of aqueous phase (for example, to vents for loading, or outlets for collection).

Sample retrieval can be conducted at different time points. In some cases, such as for cultivation of cells, the sample retrieval time point can affect the results of subsequent analysis due to differing growth rates of different cells in the sample. For example, maximum yield of biomass for a cell culture can occur in the late exponential phase of growth or the early lag phase of growth. Partitioning of samples can reduce or eliminate biases between samples, such as in yield of biomass or in amount of markers like genomic DNA. This reduction in or elimination of bias can reduce the importance of waiting for a specific time point to retrieve samples.

The initial concentration of cell in a sample can be estimated by plate count or microscopy. The cells can be encapsulated into the volume. There are a number of approaches that can be used to encapsulate cells. For example, the cells can be encapsulated by stochastic confinement. For example, the microbial suspension can first be separated into many liquid microcompartments by a process of stochastic confinement (See Meghan E. Vincent, Weishan Liu, Elizabeth B. Haney, and Rustem F. Ismagilov, “Microfluidic stochastic confinement enhances analysis of rare cells by isolating cells and creating high density environments for control of diffusible signals,” Chem. Soc. Rev. 2010 39: 974-984. DOI: 10.1039/b917851a; James Q. Boedicker, Liang Li, Timothy R. Kline, and Rustem F. Ismagilov, “Detecting bacteria and determining their susceptibility to antibiotics by stochastic confinement in nanoliter droplets using plug-based microfluidics,” Lab Chip 2008 8: 1265-1272. DOI 10.1039/b804911d; Weishan Liu, Hyun Jung Kim, Elena M. Lucchetta, Wenbin Du, and Rustem F. Ismagilov, “Isolation, incubation, and parallel functional testing and identification by FISH of rare microbial single-copy cells from multi-species mixtures using the combination of chemistrode and stochastic confinement,” Lab Chip 2009 9: 2153-2162. DOI: 10.1039/b904958d). For example, when the number of microcompartments is larger than the number of microbial cells, based on Poisson statistics, most wells can contain one or zero cells. For example, 1 nanoliter wells can be used to stochastically confine a sample at a concentration below 10⁶ or 10⁵ cells/milliliter. However, the Poisson limit can be overcome, for example when the cells can be encapsulated by actively controlled cell sorting, or when the cells can be encapsulated passively by self-organizing (See Jon F. Edd, Dino Di Carlo, Katherine J. Humphry, Sarah Koster, Daniel Irimia, David A. Weitz, and Mehmet Toner, “Controlled encapsulation of single cells into monodisperse picoliter drops,” Lab Chip August 2008, 8 (8): 1262-1264. DOI 10.1039/b805456h). Furthermore, cells can be trapped in features designed to trap the cells preferentially over other components of the sample for example, see Dino Di Carlo, Liz Y. Wu, and Luke P. Lee, “Dynamic single cell culture array,” Lab Chip, 2006, 6, 1445-1449, DOI: 10.1039/B605937F and Alison M Skelley, Oktay Kirak, Heikyung Suh, Rudolf Jaenisch, and Joel Voldman, “Microfluidic control of cell pairing and fusion,” Nature Methods 6, 147-152 (2009), DOI:10.1038/nmeth.1290).

VII. Cultivation of Organisms from Samples

The methods, compositions, and devices described in this disclosure can be used to cultivate organisms from a sample. For example, organisms from an environment can be sampled and loaded onto a device for cultivation. A sample comprising organisms can be partitioned into an original set of samples. The samples can be incubated to allow growth of colonies or populations of organisms. Subsequent to colony or population growth, the original set of samples can be split into matched sets of samples, each containing a colony or population of organisms. One set of samples can be assayed to identify those comprising the gene, activity, or other feature of interest. Corresponding samples from another set can then be selected for culture, scale-up, or other further study.

The parallel sample handling described in this disclosure can be used to isolate and cultivate targeted organisms from a sample. For example, organisms from an environment containing genes of interest can be identified and isolated for further study. In another example, organisms from an environment with an activity of interest can be identified and isolated for further study. A sample comprising organisms, of which at least one is targeted, can be partitioned into an original set of samples. The samples can be incubated to allow growth of colonies or populations of organisms. Subsequent to colony or population growth, the original set of samples can be split into matched sets of samples, each containing a colony or population of organisms. One set of samples can be assayed to identify those comprising the gene, activity, or other feature of interest. Corresponding samples from another set can then be selected for culture, scale-up, or other further study.

A sample comprising organisms can be partitioned among defined volumes. The number of cells or organisms in a given defined volume can be controlled by the initial concentration of cells or organisms in the sample being partitioned. Statistical methods can be used to determine the distribution of organisms or cells in defined volumes, such as Poisson statistics. Prior to cultivation or incubation, a defined volume can comprise at least 1 cell or organism, at least 2 cells or organisms, at least 3 cells or organisms, at least 4 cells or organisms, at least 5 cells or organisms, at least 6 cells or organisms, at least 7 cells or organisms, at least 8 cells or organisms, at least 9 cells or organisms, at least 10 cells or organisms, at least 15 cells or organisms, at least 20 cells or organisms, or at least 25 cells or organisms. Prior to cultivation or incubation, a defined volume can comprise at most 1 cell, at most 2 cells or organisms, at most 3 cells or organisms, at most 4 cells or organisms, at most 5 cells or organisms, at most 6 cells or organisms, at most 7 cells or organisms, at most 8 cells or organisms, at most 9 cells or organisms, at most 10 cells or organisms, at most 15 cells or organisms, at most 20 cells or organisms, or at most 25 cells or organisms.

A sample comprising organisms can be cultivated. Cultivation can be performed for a variety of cultivation times. Cultivation times can be at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 36 hours, or at least 48 hours. Cultivation times can be at most 30 minutes, at most 1 hour, at most 2 hours, at most 3 hours, at most 4 hours, at most 5 hours, at most 6 hours, at most 7 hours, at most 8 hours, at most 9 hours, at most 10 hours, at most 11 hours, at most 12 hours, at most 18 hours, at most 24 hours, at most 36 hours, or at most 48 hours. Cultivation can be performed at a variety of temperatures. Cultivation temperatures can be at least −10° C., at least −5° C., at least 0° C., at least 5° C., at least 10° C., at least 15° C., at least 20° C., at least 25° C., at least 30° C., at least 35° C., at least 37° C., at least 40° C., at least 45° C., at least 50° C., at least 55° C., at least 60° C., or at least 65° C. Cultivation temperatures can be at most −10° C., at most −5° C., at most 0° C., at most 5° C., at most 10° C., at most 15° C., at most 20° C., at most 25° C., at most 30° C., at most 35° C., at most 37° C., at most 40° C., at most 45° C., at most 50° C., at most 55° C., at most 60° C., or at most 65° C. Cultivation can be performed at room temperature. Cultivation can be performed at 37° C.

VIII. Assays and Identification Methods

Organisms and other samples of interest can be identified by a variety of means. Identification can be conducted on while the sample is still within the parallel sample handling system (e.g. SlipChip device, microfluidic droplet device, microwell array, or other parallel sample handling system). Microfluidic droplet devices are described, for example, in U.S. Pat. No. 7,129,091, incorporated by reference herein in its entirety. Identification can be conducted on samples removed from the parallel sample handling system.

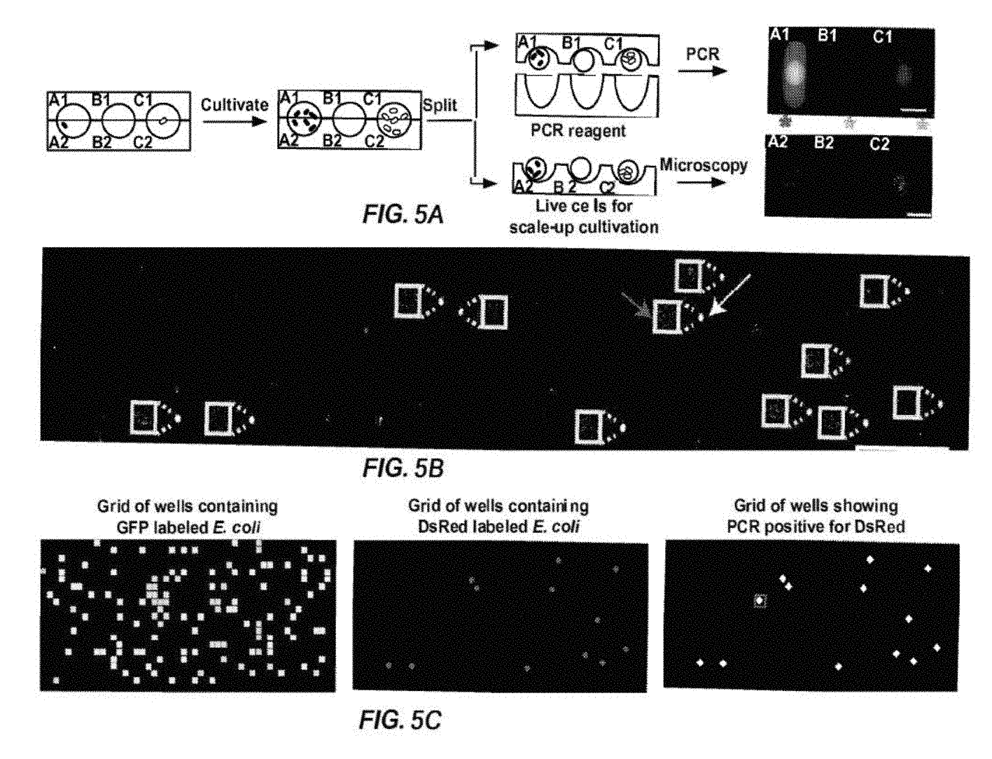

Organisms of interest can be selected or identified by genetic markers. Identification by genetic markers can be conducted by PCR. PCR primers targeting a genetic region of interest (e.g. genes, sets of genes, promoters, or gene constructs) can be used to conduct PCR, and the presence of PCR product indicates the presence of the genetic region of interest. For example, organisms can be dispersed into original sample volumes on a SlipChip, cultivated, and split FIG. 5A). One plate of the SlipChip can be combined with a SlipChip plate comprising PCR reagent and reacted to form PCR product (FIG. 5B), with the presence of PCR product indicating the presence of the genetic region of interest. Isothermal amplification methods can be used. Identification of genetic markers can be conducted by FISH. Fluorescent probes targeting a genetic region of interest can be added to samples and allowed to hybridize. Unbound probes can be washed away, and the presence of fluorescent probes indicates the presence of the genetic region of interest. Identification of genetic markers can be by sequencing. A single nucleic acid molecule from each sample can be sequenced. Nucleic acid molecules from each sample can be amplified, and the amplification products can be sequenced. Amplification and sequencing can target specific regions or can target the entire genome.

A function-targeted approach can also be used. Functional assays can share the general workflow developed in the gene-based method. Assays including (but not limited to) fluorescent, colorimetric, chemiluminescent, or mass spectrometry assays, can be used to identify microbes that can perform a specific function. Such high throughput functional assays can also be performed directly on clinical samples, if desired. The microbial cell microarray created from splitting the SlipChip, for example, can be combined with a piece of biological tissue (such as, for example, from an intestine) to perform functional assays. In some cases, the tissue from an intestine can be obtained from transgenic mice with a GFP reporter for the function of interest. After the tissue is combined with the microbial cell array and incubated, localized GFP expression can be visualized by using standard epifluorescence microscopy. If desired, corresponding colonies can then be retrieved from the second plate for another purpose, such as a scale-up culture of isolates of interest.

In some cases, the functional activity being identified or targeted is enzymatic. The functional activity can generate a visible or UV-absorbing product. The enzymatic method can be the CHOD-PAP method for detecting cholesterol oxidase activity. The enzymatic method can be the GOD-Perid method for detecting glucose oxidase activity. The enzymatic method can be for detecting lactate dehydrogenase activity. The enzymatic activity can be the degradation of cellulose, using a cellulose degradation dye. The enzymatic activity can be the degradation of lignin, using a lignin degradation dye. In some cases, the functional activity is detected by interaction with a fluorogenic substrate. For example, N,N-dibutyl phenylene diamine can be used to detect H₂S to detect the activity of a sulfate-reducing organism.

In some cases, the functional activity being identified or targeted is a resistance or set of resistances. The resistance can be to antibiotics. The resistance can be to particular growth conditions, such as temperature, atmosphere, pressure, presence of other organisms, or other conditions. The identification can be based on the presence, absence, magnitude, or rate of growth under the selected conditions.

In some cases, the functional activity being identified is response to a chemical. The response can be negative, for example inhibition of growth. The response can be positive, for example promotion of growth. The identification can be based on the presence, absence, magnitude, or rate of growth under the selected conditions.

IX. Gas Control

While liquid cultivation medium can be important to support the growth of microbes, controlling the content of the gas phase can also be important for microbial cultivation. Some microbes may not be able to grow due in the presence of certain gases. For example, obligate anaerobes are sensitive to oxygen and would not grow upon exposure to air. Some microbes require a certain type of gas to grow. For example, most archaea are autotrophs that would assimilate CO₂ into cellular material, and would not grow in the absence of CO₂. Accumulation of waste product such as hydrogen from thermotogales or hydrogen sulfide from sulfate reducing bacteria can be inhibitory for their growth.

Gas control can be achieved by locating devices within a chamber with a controlled environment, such as an anaerobic chamber. For example, in the context of anaerobic cultivation, the partial pressure of oxygen in the gas phase is usually controlled by an anaerobic chamber or the Hungate roll tube technique. Oxygen in the gas phase of the anaerobic chamber can be reduced by hydrogen with palladium catalyst. This method is compatible with cultivating most microbes from the human gut, and allows the use of agar plates. The Hungate roll tube method is widely used to cultivate more strict anaerobes such as methanogens, where the use of a glass tube and butyl rubber stopper can effectively prevent diffusion of oxygen into the vessel. Any type of environment, which can be a container or bottle which can be sealed, can be used to provide an environment for cultivation. For example, devices can be placed within a glass bottle, into which the desired gas or gases can then be injected.

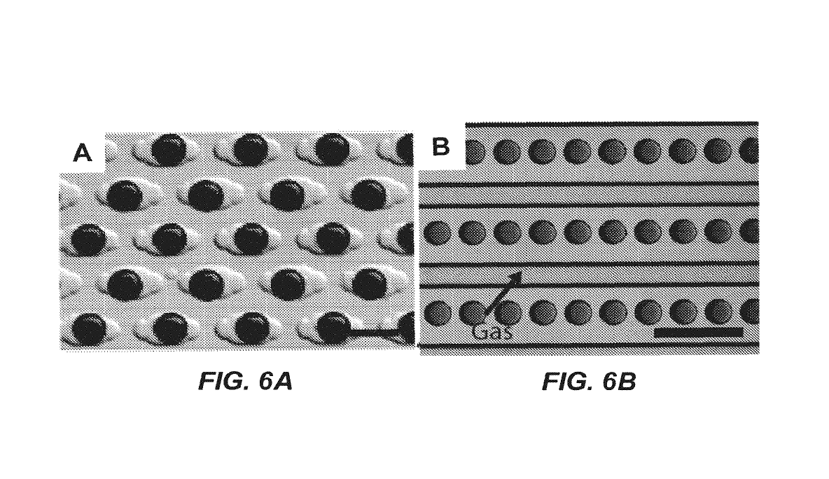

Gas control can be achieved by incorporating gas supply channels onto devices themselves. For example, a SlipChip device (FIG. 6A) can have wells with oil and aqueous phases. A SlipChip device can be designed to include gas supply channels (FIG. 6B). The distance between the channels and the wells can be on the order of hundreds of micrometers, resulting in a characteristic diffusion time for oxygen through the device substrate on the order of minutes.

Gas control can be achieved by increasing the size of the gap between components of the device. A SlipChip device with facing plates can have the distance between the plates increased by the addition or fabrication of spacers or posts. For example, plate substrate can be etched to create post features to increase the gap distance between the plates. Alternatively, posts or spacers can be fabricated by the addition of material, such as metal, plastic, oxide, or photoresist, to the plate surface. Post or spacer height can be less than or equal to about 100 nm, less than or equal to about 200 nm, less than or equal to about 400 nm, less than or equal to about 500 nm, less than or equal to about 700 nm, less than or equal to about 900 nm, less than or equal to about 1.5 μm, or less than or equal to about 2 μm. Post height can be greater than or equal to about 100 nm, greater than or equal to about 200 nm, greater than or equal to about 400 nm, greater than or equal to about 500 nm, greater than or equal to about 700 nm, greater than or equal to about 900 nm, greater than or equal to about 1.5 μm, or greater than or equal to about 2 μm. Spacers or posts can be fabricated by wet chemical etching, such as with hydrofluoric acid. Spacers or posts can be fabricated by plasma etching, such as by reactive ion etching or deep reactive ion etching. The fabrication of spacers or posts can be conducted with photolithography, soft lithography, laser ablation, micromolding, embossing, or other microfabrication techniques. The gap can allow increased gas transfer from the outside atmosphere, from on-chip gas supply channels, or both. Cell growth can vary based on the gas transfer enabled by different gap distances.

Gas control can be achieved by selection of device materials. Different device materials, such as glass, PDMS, PMMA, other plastics, and metals, permit different rates of diffusion through the substrate. If an oil phase is used, different oils, such as perfluorinated oil, mineral oil, or other oils, permit different rates of diffusion.

Gases controlled can comprise a wide range of single gases or combinations of gases. Examples of gases include O₂, CO₂, CO, N₂, NO, NO₂, H₂O, air, or other gases. Gases controlled can be provided at different pressures, including atmospheric pressure, higher than atmospheric pressure, or lower than atmospheric pressure.

X. Chemical Communication Control

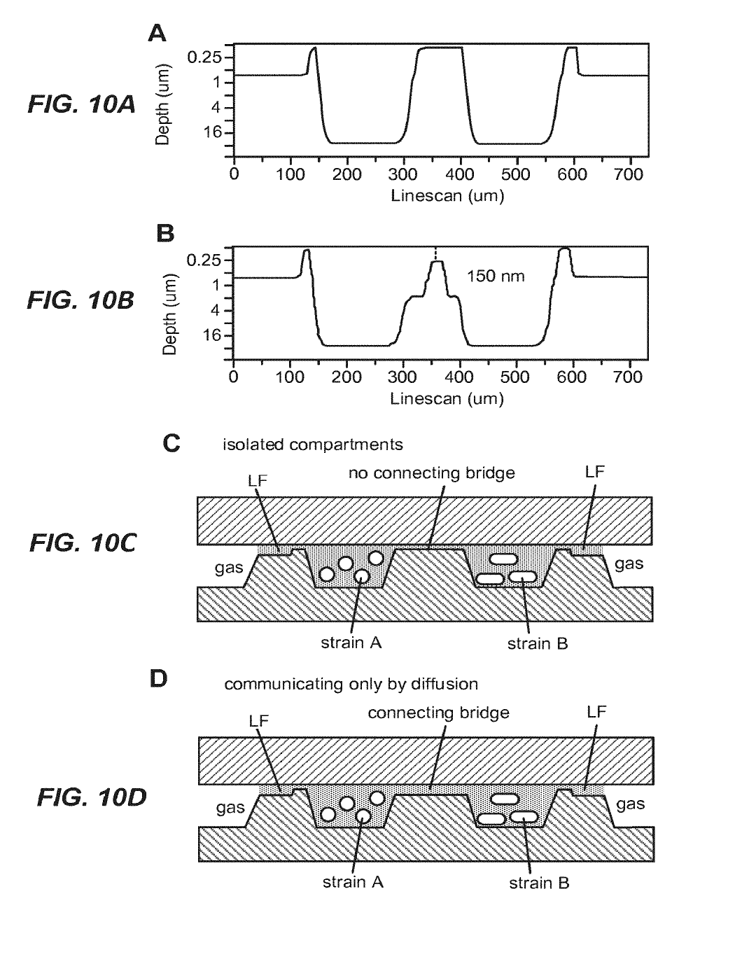

Devices can be designed to contain analytes within defined volumes while permitting chemical communication between defined volumes. For example, a pair of wells can be connected by a bridge (FIG. 10B, FIG. 10D) permitting diffusive chemical connection while keeping cells contained in their respective wells, rather than the contents of each well being completely isolated (FIG. 10A, FIG. 10C).

XI. On Chip Dilution

Dilution can be performed on-chip. Dilution can be linear. Dilution can be logarithmic. Dilution can comprise a single dilution step. Dilution can comprise multiple dilution steps.

Dilution can be performed with a SlipChip device. Dilution can be performed by actuating a SlipChip to bring volumes of sample in contact with volumes of diluent. Multiple steps of dilution can be performed by actuating a SlipChip multiple times, to bring volumes of sample in contact with a series of volumes of diluent. The SlipChip well surfaces can be hydrophilic or hydrophobic. Dilution can be performed on one device or on multiple devices.

Mixing can occur between dilution steps. Mixing can be active, by an applied mixing force such as sonication, vortexing, agitation, stirring, electrohydrodynamic forces, generation of flow within a volume, or other means. Mixing can be passive, such as by allowing adequate time for diffusive mixing. The volumes of sample and the volumes of diluent can be equal in volume, or can differ in volume.

XII. Automation

The methods, devices, and systems provided in this disclosure can be used with automation equipment or robotics. Fabrication of systems and devices for parallel sample handling, such as SlipChip devices, can be automated. Pre-treatment and loading of samples onto devices can be automated. Parallelization of samples by splitting original samples can be automated. Slipping or actuation of SlipChip components to actuate on-device operations, such as mixing, splitting, diluting, allowing chemical communication between can be automated. Cultivation or amplification of samples can be automated. Splitting of SlipChip plates to produce parallelized samples can be automated. Conducting assays or other techniques to identify defined volumes containing samples of interest can be automated. Retrieving samples of interest, or conducting a chip wash to retrieve and pool all samples, can be automated. Controlling the sample environment, including gas atmosphere, temperature, and other parameters can be automated. Identification of matched sets of samples on different substrates can be automated, for example using computerized image analysis methods.

XIII. Pathogen Diagnostics

The methodologies and devices here can be used for detecting, quantifying, or analyzing pathogenic microorganisms and diagnosing conditions associated with these pathogens. Examples of bacterial pathogens include but arc not limited to Acromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formerly Pseudomonas mallei); Burkholderia pseudomallei (formerly Pseudomonas pseudomallei); Campylobacter jejuni; Chlamydia psittaci; Clostridium botulinum; Clostridium botulinum; Clostridium perfringens; Coccidioides immitis; Coccidioides posadasii; Cowdria ruminantium (Heartwater); Coxiella burnetii; Enterovirulent Escherichia coli group (EEC Group) such as Escherichia coli—enterotoxigenic (ETEC), Escherichia coli—enteropathogenic (EPEC), Escherichia coli—O157:1-17 enterohemorrhagic (EHEC), and Escherichia coli—enteroinvasive (EIEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacteriumtuberculosis; Mycoplasma capricolum; Mycoplasma mycoides ssp mycoides; Peronosclerospora philippinensis; Phakopsora pachyrhizi; Plesiomonasshigelloides; Ralstonia solanacearum race 3, biovar 2; Rickettsia prowazekii; Rickettsia rickettsii; Salmonella spp.; Schlerophthora rayssiae varzeae; Shigella spp.; Staphylococcus aureus; Streptococcus; Synchytriumendobioticum; Vibrio cholerae non-O1; Vibrio cholerae O1; Vibrioparahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis.

Further examples of organisms include viruses such as: Africanhorse sickness virus; African swine fever virus; Akabane virus; Avianinfluenza virus (highly pathogenic); Bhanja virus; Blue tongue virus (Exotic); Camel pox virus; Cercopithecine herpesvirus 1; Chikungunya virus; Classicalswine fever virus; Coronavirus (SARS); Crimean-Congo hemorrhagic fevervirus; Dengue viruses; Dugbe virus; Ebola viruses; Encephalitic viruses such as Eastern equine encephalitis virus, Japanese encephalitis virus, Murray Valley encephalitis, and Venezuelan equine encephalitis virus; Equinemorbillivirus; Flexal virus; Foot and mouth disease virus; Germiston virus; Goat pox virus; Hantaan or other Hanta viruses; Hendra virus; Issyk-kul virus; Koutango virus; Lassa fever virus; Louping ill virus; Lumpy skin disease virus; Lymphocytic choriomeningitis virus; Malignant catarrhal fever virus (Exotic); Marburg virus; Mayaro virus; Menangle virus; Monkeypox virus; Mucambovirus; Newcastle disease virus (WND); Nipah Virus; Norwalk virus group; Oropouche virus; Orungo virus; Peste Des Petits Ruminants virus; Piry virus; Plum Pox Potyvirus; Poliovirus; Potato virus; Powassan virus; Rift Valley fevervirus; Rinderpest virus; Rotavirus; Semliki Forest virus; Sheep pox virus; South American hemorrhagic fever viruses such as Flexal, Guanarito, Junin, Machupo, and Sabia; Spondweni virus; Swine vesicular disease virus; Tick-borne encephalitis complex (flavi) viruses such as Central European tick-borne encephalitis, Far Eastern tick-borne encephalitis, Russian spring and summer encephalitis, Kyasanur forest disease, and Omsk hemorrhagic fever; Variola major virus (Smallpox virus); Variola minor virus (Alastrim); Vesicularstomatitis virus (Exotic); Wesselbron virus; West Nile virus; Yellow fever virus; and South American hemorrhagic fever viruses such as Junin, Machupo, Sabia, Flexal, and Guanarito.

Further examples of organisms include parasitic protozoa and worms, such as: Acanthamoeba and other free-living amoebae; Anisakis sp. and other related worms Ascaris lumbricoides and Trichuris trichiura; Cryptosporidium parvum; Cyclospora cayetanensis; Diphyllobothrium spp.; Entamoeba histolytica; Eustrongylides sp.; Giardia lamblia; Nanophyetus spp.; Shistosoma spp.; Toxoplasma gondii; Filarial nematodes and Trichinella. Further examples of analytes include allergens such as plant pollen and wheat gluten.

Further examples of organisms include fungi such as: Aspergillus spp.; Blastomyces dermatitidis; Candida; Coccidioides immitis; Coccidioides posadasii; Cryptococcus neoformans; Histoplasma capsulatum; Maize rust; Rice blast; Rice brown spot disease; Rye blast; Sporothrixschenckii; and wheat fungus.

XIV. Microbiome Diagnostics and Isolation

The methodologies and devices described here can be useful for personalized diagnostics of human disease based on the composition and function of the microbiome. A number of disease affecting millions of Americans such as IBD, infections, diabetes, autoimmune conditions, and obesity have connections to the microbiome. Other processes such as Allergies, Diarrhea, Lactose intolerance, Control of Cholesterol levels, Control of blood pressure, Immune function and infections, Helicobacter pylori, Inflammation, Bacterial growth under stress, Irritable bowel syndrome and colitis, HIV infection and other viral infections, Necrotizing enterocolitis, Vitamin production, Eczema, Bacterial Vaginosis, Drug metabolism and side effects, Clostridium Difficile infection, autism and other complex nervous system disorders may also be correlated with the microbiome. The healthy or diseased state of the host can be classified using taxonomic or functional profile (See Shi Huang, Rui Li, Xiaowei Zeng, Tao He, Helen Zhao, Alice Chang, Cunpei Bo, Jie Chen, Fang Yang, Rob Knight, Jiquan Liu, Catherine Davis and Jian Xu, “Predictive modeling of gingivitis severity and susceptibility via oral microbiota,” ISME J advance online publication, Mar. 20, 2014; doi:10.1038/ismej.2014.32 [Epub ahead of print] and Nicola Segata, Jacques Izard, Levi Waldron, Dirk Gevers, Larisa Miropolsky, Wendy S Garrett, and Curtis Huttenhower, “Metagenonmic biomarker discovery and explanation,” Genom Biol. 2011; 12 6); R60; doi: 10.1186/gb-2011-12-6-r60). This can be done by obtaining a sample containing live organism from the subject, distributing this sample over a microfabricated substrate, enabling growth of at least one microorganism including bacteria, fungi, archaea and viruses, and protozoa, performing genetic or functional assays to detect the relative and/or absolute abundance of the marker taxa or function, and using this information to determine, or to predict at a later stage, the health and disease state of the host. In some cases, this method and device can also be used to determine the history of the host, and can be useful for forensic applications (See Noah Fierer, Christian L. Lauber, Nick Zhou, Daniel McDonald, Elizabeth K. Costello, and Rob Knight, “Forensic identification using skin bacterial communities,” Proceedings of the National Academy of Sciences of the United States of America, 2010 Apr. 6; 107(14):6477-81. doi: 10.1073/pnas.1000162107).

Isolation of microbes by the methods described herein can improve quality of life and reduce healthcare costs by modulating dysbiosis of the human gut in therapeutic and prophylactic modes (See Elaine O Petrof, Gregory B Gloor, Stephen J Vanner, Scott J Weese, David Carter, Michelle C Daigneault, Eric M Brown, Kathleen Schroeter, and Emma Allen-Vercoe, “Stool substitute transplant therapy for eradication of Clostridium difficile infection: ‘RePOOPulating’ the gut,” Microbiome. 2013 Jan. 9; 1(1):3. doi: 10.1186/2049-2618-1-3) in the context of conditions affecting millions of people, including but not limited to IBD, infections, diabetes, autoimmune conditions, and obesity. A number of conditions have connections to the microbiome and can be treated therapeutically and/or prophylactically with microbes and microbial communities isolated using the methodology described herein. These include but are not limited to Allergies, Diarrhea, Lactose intolerance, Control of Cholesterol levels, Control of blood pressure, Immune function and infections, Helicobacter pylori, Inflammation, Bacterial growth under stress, Irritable bowel syndrome and colitis, HIV infection and other viral infections, Necrotizing enterocolitis, Vitamin production, Eczema, Bacterial Vaginosis, Drug metabolism and side effect, Clostridium Difficile infection, autism and other complex nervous system disorders. This methodology can be used for the amelioration, stabilization, treatment and/or prevention of, or decreasing or delaying the symptoms of, an infection, disease, treatment, poisoning or a condition having a bowel dysfunction component or side-effect, or for the amelioration, treatment and or prevention of a constipation, for the treatment of an abdominal pain, a non-specific abdominal pain or a diarrhea, a diarrhea caused by: a drug side effect or a psychological condition or Crohn's Disease, a poison, a toxin or an infection, a toxin-mediated traveler's diarrhea, or a Clostridium or a C. perfringens welchii or a C. difficile infection or a pseudo-membranous colitis associated with a Clostridium infection, or for preventing, or decreasing or delaying the symptoms of, or ameliorating or treating individuals with spondyloarthropathy, spondylarthritis or sacroileitis (an inflammation of one or both sacroiliac joints); a nephritis syndrome; an inflammatory or an autoimmune condition having a gut or an intestinal component; lupus; irritable bowel syndrome (IBS or spastic colon); or a colitis; Ulcerative Colitis or Crohn's Colitis; constipation; autism; a degenerative neurological diseases; amyotrophic lateral sclerosis (ALS), Multiple Sclerosis (MS) or Parkinson's Disease (PD); a Myoclonus Dystonia; Steinert's disease; proximal myotonic myopathy; an autoimmune disease; Rheumatoid Arthritis (RA) or juvenile idiopathic arthritis (HA); Chronic Fatigue Syndrome; benign myalgic encephalomyelitis; chronic fatigue immune dysfunction syndrome; chronic infectious mononucleosis; epidemic myalgic encephalomyelitis; obesity; hypoglycemia, pre-diabetic syndrome, type I diabetes or type II diabetes; Idiopathic thrombocytopenic purpura (ITP); an acute or chronic allergic reaction; hives, a rash, a urticaria or a chronic urticaria; and/or insomnia or chronic insomnia, Grand mal seizures or petit mal seizures. Both human and animal conditions can be treated therapeutically or prophylactically using the methodology described herein.

Major changes in enteric microbiota significantly decrease people's duration and quality of life, such as in colorectal cancer (A. C. Society Colorectal Cancer Facts and FIGS. 2008-2010; American Cancer Society: Atlanta, 2008.) and in inflammatory bowel disease (IBD), which alone affects up to 1.4 million people in the United States (E. V. Loftus. Clinical epidemiology of inflammatory bowel disease: Incidence, prevalence, and environmental influences. Gastroenterolgy. 2004 126 1504-1517). Inflammatory Bowel Disease (IBD) includes both Ulcerative Colitis and Crohn's Disease. Furthermore, diabetes, obesity, and autoimmune disorders have also been linked to changes in intestinal microbes (L. Wen, R. E. Ley, P. Y. Volchkov, P. B. Stranges, L. Avanesyan, A. C. Stonebraker, C. Hu, F. S. Wong, G. L. Szot, J. A. Bluestone, J. I. Gordon and A. V. Chervonsky. Innate immunity and intestinal microbiota in the development of Type 1 diabetes. 2008 455 1109-1113). Undesirable changes of the microbiome include loss of beneficial communities—for example, those producing butyrate, a signal that regulates gene expression of the host's epithelium and serves as its main nutrient—and blooms of pathogenic microbes such as sulfur-reducing bacteria that produce hydrogen sulfide (H₂S), a potent signal that profoundly affects the host. The use of current probiotic mixtures has had surprisingly limited success in clinical trials (Vogel, G. Clinical trials. Deaths prompt a review of experimental probiotic therapy. Science 319, 557 (2008). O'Mahony, L., Feeney, M., O'Halloran, S., Murphy, L., Kiely, B., Fitzgibbon, J., Lee, G., O'Sullivan, G., Shanahan, F. & Collins, J. K. Probiotic impact on microbial flora, inflammation and tumour development in IL-10 knockout mice. Aliment Pharmacol Ther 15, 1219-1225 2001).

Microbes can be isolated from a body site, such as skin or gut, or from a bodily sample such as a stool, saliva, or a genital swab sample. Microbes can be isolated from the patient or from a donor matched to the patient in one or more characteristics, such as genetic profile, age, gender, medical history, diet, or environment. Microbial isolation can be done pre-emptively (before the disease developed), with microbes optionally preserved for future use. Microbes can be isolated at any stage of disease progression. Microbes that can be isolated for these purposes include bacteria, fungi, archaea and viruses, protozoa. Microbes can also be isolated from an environment including soil environment, built environment, marine environment.

Isolation of microbes can be guided by genetic assays. For example, a marker gene (such as 16S RNA gene) associated with a particular species or genus of microbes can be used to target the isolation (Schloss P, Handelsman J (2005) Metagenomics for studying unculturable microorganisms: Cutting the gordian knot. Genome Biology 6(8):229. Fodor A A, DeSantis T Z, Wylie K M, Badger J H, Ye Y, Hepburn T, Hu P, Sodergren E, Liolios K, Huot-Creasy H, Birren B W, Earl A M 2012) The “most wanted” taxa from the human microbiome for whole genome sequencing. PLoS ONE 7(7):e41294. Kennedy J, O'Leary N D, Kiran G S, Morrissey J P, O'Gara F, Selvin J, Dobson A D W (2011) Functional metagenomic strategies for the discovery of novel enzymes and biosurfactants with biotechnological applications from marine ecosystems. J. Appl. Microbiol. 111(4):787-799. Rooks D J, McDonald J E, McCarthy A J (2012) Chapter twenty—metagenomic approaches to the discovery of cellulases. Methods in enzymology, ed Harry J G (Academic Press), Vol Volume 510, pp 375-394. Reddy B V B, Kallifidas D, Kim J H, Charlop-Powers Z, Feng Z, Brady S F (2012) Natural product biosynthetic gene diversity in geographically distinct soil microbiomes. Applied and Environmental Microbiology 78(10):3744-3752. Ridaura V K, Faith J J, Rey F E, Cheng J, Duncan A E, Kau A L, Griffin N W, Lombard V, Henrissat B, Bain J R, Muehlbauer M J, Ilkayeva O, Semenkovich C F, Funai K, Hayashi D K, Lyle B J, Martini M C, Ursell L K, Clemente J C, Van Treuren W, Walters W A, Knight R, Newgard C B, Heath A C, Gordon J I (2013) Gut microbiota from twins discordant for obesity modulate metabolism in mice. Science 341(6150). Frank D N, St. Amand A L, Feldman R A, Boedeker E C, Harpaz N, Pace N R (2007) Molecular-phylogenetic characterization of microbial community imbalances in human inflammatory bowel diseases. Proceedings of the National Academy of Sciences 104(34):13780-13785.). For example, a functional gene (for example, a gene associated with carbohydrate degradation, mucoadhesion, short chain fatty acid production, production of lipopolysaccharides such as polysaccharide A, PSA, vitamin production, production of anti-inflammatory compounds, production of non-ribosomal peptides, production of polyketide natural products) can be used to target the isolation and cultivation of the organism. Furthermore, functional assays can be used, including assays for enzymatic activity.

Target genes can be determined by metagenomic analysis using, for example, high-throughput sequencing technologies. Other organisms, such as nematodes and similar organisms can be targeted. Simple technologies for personalized bacterial isolation in a clinical setting may create opportunities for improving ability to diagnose disease and lead to the development of probiotic treatments with enhanced prophylactic and therapeutic properties.

This methodology uses bacteriotherapy to displace pathogenic or undesired organisms in the patient with healthy or desirable microbiota.

Fecal microbiota transplantation can be used for treating diseases (such as, for example Clostridium difficile infection). Fecal microbiota transplantation delivers the fecal material from a healthy donor to patient to displace the undesired organisms in the gut microbiota. However, this method can be risky as pathogens may be present in the fecal material transplanted and can be dangerous for the immunocompromised patients. Each individual has a personalized gut microbiota that may contain healthy or desirable gut microbiota. A sample containing live organisms can be obtained from a person. Personal probiotics can be developed using this methodology. In one example, organisms may be obtained from the individual subject or patient prior to the disease state, isolated and stored, and delivered back to the same subject and used to treat disease or improve the health state of the individual. Organisms can also be obtained from relatives and administered to the patient. Organisms obtained from relatives may be beneficial for patients due to matches in genetics, diet and living habits. Organisms could also be obtained from donors that are matched to the patient in some way, e.g. racial, genetic, diet, body mass index and other parameters. Any combination of organisms obtained from the patient, relatives, or other donors could be used with this methodology to treat disease or improve the health state of patients.

Culture-independent techniques can provide insights into microbial ecology by revealing genetic signatures of a person's microorganisms. It can suggest that certain microbes may impact host phenotypes such as obesity, inflammation, and gastrointestinal integrity. Data sets from high-throughput sequencing suggest microbial targets with high biomedical importance. These targets can include, but are not limited to, Eubacterium limosum, Roseburia intestinalis, F. prausnitzii, Roseburia spp., Eubacterium rectale, B. ovatus, P. distasonis, Eubacterium eligens, Eubacterium ventriosum, Roseburia spp., Blautia spp., Dorea spp., R. torques, Bifidobacterium longum, Eubacterium hadrum, Anaerostipes coli, Clostridium spp. aldenense, Clostridium spp. hathewayi, symbiosum, orbiscindens, thermocellum, citroniae, Ruminococcus obeum, Ruminococcus productus, Ruminococcus torques, Ruminococcus bromii, Roseburia inulinovorans, Blautia coccoides, Dorea spp., Sutterella spp., Dialister invisus, Blautia producta, and Bifidobacterium pseudocatenulatum. These targets can also be fungi, archaea, viruses or protozoa. The method described herein can be used for genetically targeted isolation and cultivation of these microorganisms from clinical samples. The sample, which may be from the patient, a relative, and/or other donor, can distributed over a microfabricated substrate containing volumes. Growth of at least one target microorganism can be achieved by using one or both of the following two methods: (i) identification of cultivation conditions for microbes using growth substrates available only in small quantities as well as the correction of sampling bias using a “chip wash” technique; and ii) performing on-chip genetic assays while also preserving live bacterial cells for subsequent scale-up cultivation of desired microbes, enabled by splitting technology to create arrays of individually addressable replica microbial cultures. One or more of the target microorganism can be isolated using this method. At least one of the said microorganisms of interest can be delivering to a human with the intent to prevent, cure, or treat a disease/with the intent to improve health. For example, one way to achieve it is through colonoscopy. For example, antibiotic therapy can be withheld (for example, for two days) and the patients can undergo standard colon cleansing the evening prior to colonoscopy. The following morning during colonoscopy, one-half (for example 50 mL) of the solution containing at least one of the said microorganisms of interest can be deposited in the region of the cecum/proximal ascending colon and the other half can be drizzled throughout the transverse colon as the colonoscope is withdrawn. Patients can be instructed to eat a fiber-rich diet and not to consume products containing probiotics. Patients can be followed by a study nurse to obtain stool samples and closely monitor their clinical response.

XV. Applications

The methods and compositions described in this disclosure can be used for the identification of organisms of interest. Samples can be dispersed among defined volumes and cultivated. Samples can be parallelized, and one set of the parallelized samples can be analyzed to identify samples containing organisms of interest. Analysis can be by a variety of methods, as described in this application. Upon identification of samples of interest, corresponding samples from the second set of parallelized samples can be selected.

The methods and compositions described in this disclosure can be used for the identification of growing conditions for organisms. Samples can be dispersed among defined volumes on multiple devices. Each device can be subjected to different conditions, such as growth media, temperature, gas atmosphere, and/or pressure. After cultivation under the various conditions, samples can be parallelized and those samples exhibiting growth under a given set of conditions can be identified through analysis of one set of parallelized samples.

The methods and compositions described in this disclosure can be used for genetic analysis. For example, a sample containing nucleic acids can be dispersed among defined volumes on a device for digital PCR. Digital PCR can be conducted, and the samples can be parallelized. Those samples with positive signals can have one set of parallelized samples used for sequencing.

The methods and compositions described in this disclosure can be used for study or rapid diagnosis of mastitis (bovine or human). A sample can be taken from breast tissue, milk, or other sources and dispersed among defined volumes and cultivated. Samples can be parallelized, and one set of the parallelized samples can be analyzed to identify organisms associated with mastitis. Cultivation can be conducted in the presence of antibiotics to test for antibiotic susceptibility.

The methods and compositions described in this disclosure can be used for study, rapid growth, detection, and/or identification of sulfate reducing organisms and other organisms leading to pipeline corrosion. A sample can be taken from a pipeline, an gut of an organism, or another source, dispersed among defined volumes, and cultivated. Samples can be parallelized, and one set of the parallelized samples can be analyzed to identify organisms associated with sulfate reduction or pipeline corrosion.

Single cells or a number of cells in a mixed community of microorganisms (for example bacteria, archaea, fungi, viruses, protozoa, or others as described herein) from a sample (such as an environmental or clinical sample) can be introduced into the device and encapsulated into a volume. Genetic material (for example DNA or RNA) can be amplified inside the volume (for example, using multiple displacement amplification or PCR, RT-PCR, or isothermal amplification). The two substrates can be separated to retrieve the amplified material. This method can be used for single cell genomics and transcriptomics.

### EXAMPLES

### Example 1—Gas Control for B. Theta

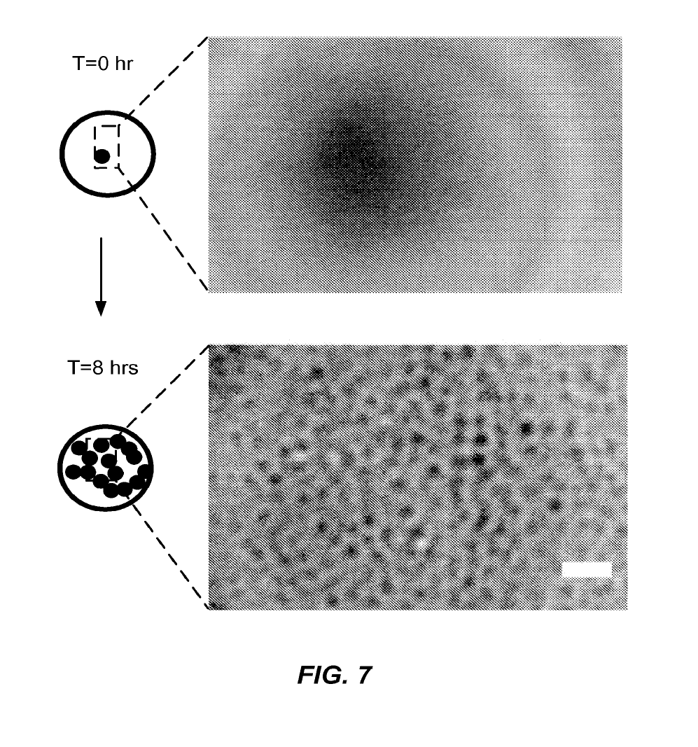

SlipChip devices with wells for cell culture were fabricated with a glass substrate. Bacteroides thetaiotaomicron B. theta) and Cooked Meat Medium cell culture medium were loaded onto the devices inside an anaerobic chamber. The devices were sealed and removed for imaging, then returned to the chamber for incubation. Devices were incubated for 8 hours at 37° C. B. theta cells grew to a dense micro-colony (FIG. 7).

### Example 2—Gas Control Via Nanoposts for E. coli

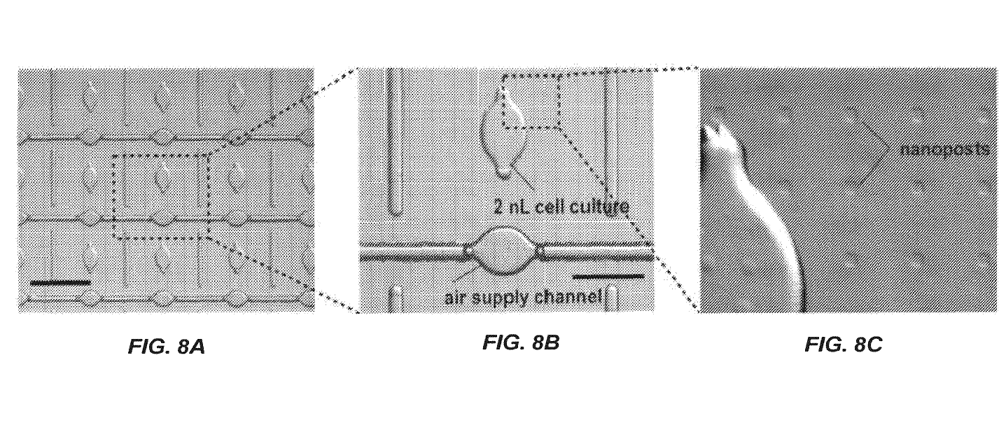

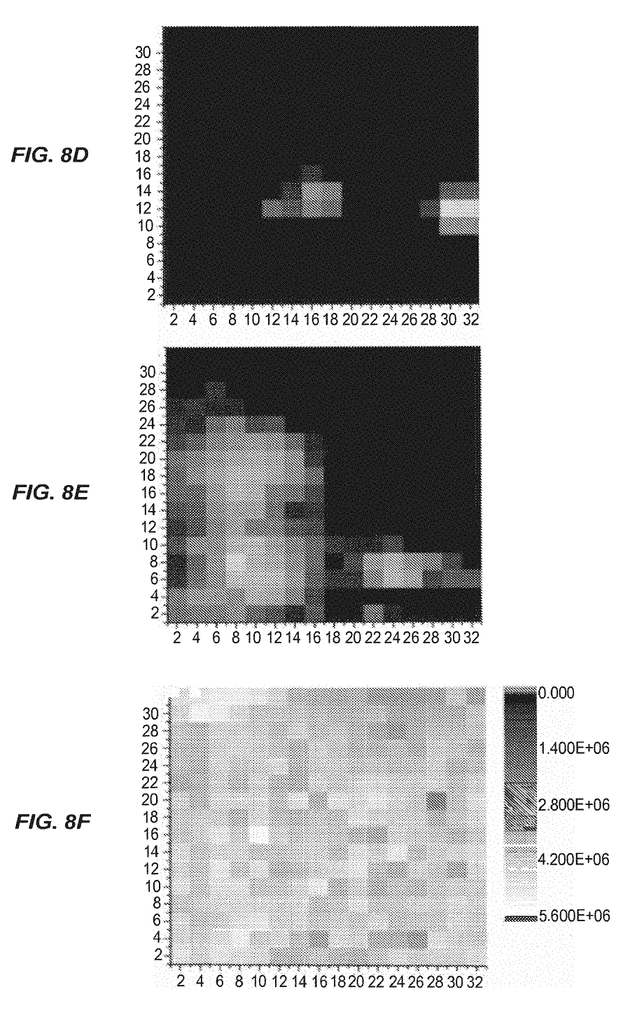

SlipChip devices with chambers for cell culture were fabricated (FIG. 8A B). Sub-micron scale nano-posts (FIG. 8C) were fabricated on some devices by immersion in diluted buffered hydrofluoric acid (HF). A fluorescently labeled strain of E. coli was loaded on devices with no nanoposts, 400 nm nanoposts, and 900 nm nanoposts, respectively (FIG. 8D F), and integrated fluorescence intensity was used to quantify growth. In this particular model system, the growth of E. coli was limited by the supply of oxygen. By tuning the gap between the two glass plates and thus controlling the gas exchange through the oil phase, a more uniform growth of E. coli was achieved.

### Example 3—Gas Control Via Sealed Vessel

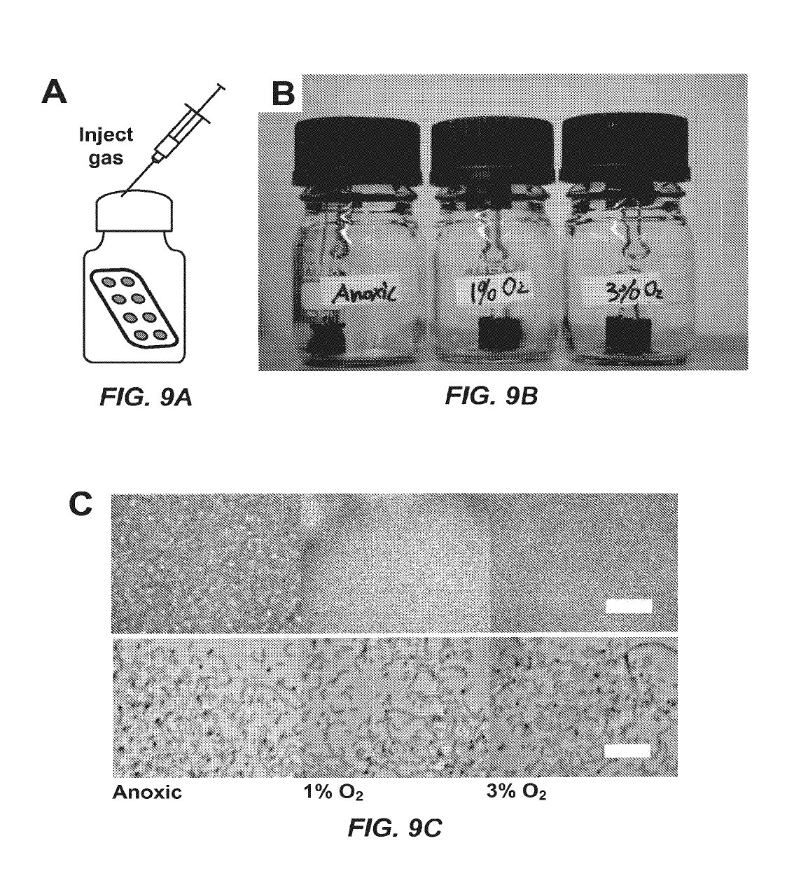

SlipChip devices containing 1600 wells with 6 nL per well were designed and fabricated so to fit into 100-mL Corning glass bottles (FIG. 9A B). Devices were loaded with cells and medium, placed in bottles, and gas mixtures with varying amounts of oxygen (O %, 1%, and 3% O₂) were injected into the bottles. Two model microorganisms were cultivated in this setup. A strict anaerobe species, B. theta was cultivated to test for the presence of oxygen in the anoxic bottle. The growth of B. theta in the 0% oxygen bottle (FIG. 9C, first column of top row) confirmed that the vessel is well sealed. B. theta was not able to grow when oxygen was injected. As a positive control, E. faecalis was grown under these three conditions (FIG. 9C, bottom row). E. faecalis grew under all three conditions.

### Example 4—on Chip Co Culture with A. Caccae and B. Theta

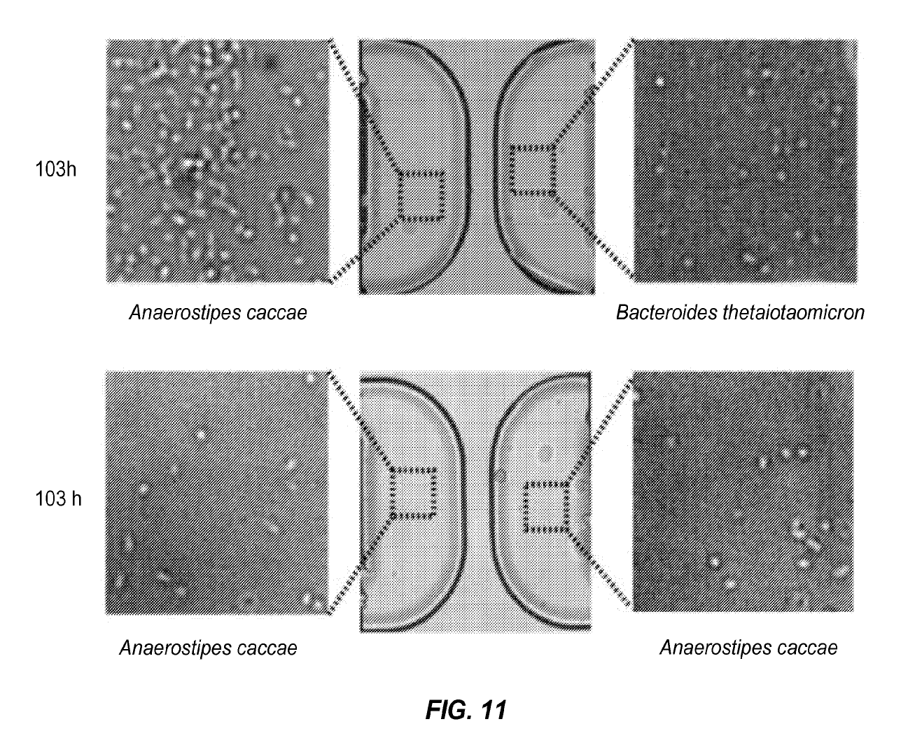

A SlipChip device for co-culture was designed as shown in FIG. 10. This device provides nanometer-deep hydrophilic bridges that allow chemical communication between microwells by diffusion, but are narrow enough to prevent mixing of microbial cells. The anaerobe Anaerostipes caccae A. caccae) cultured in minimal media with inulin as the sole carbon source grows when co-cultured with B. theta, but not when cultured alone. A. caccae and B. theta were loaded into diffusively-connected wells of the SlipChip device and cultured with minimal media. For comparison, A. caccae was loaded into both of a pair of diffusively-connected wells and cultured with minimal media. Wells containing A. caccae diffusively connected to wells containing B. theta exhibited significantly more growth (FIG. 11, top) than wells containing A. caccae diffusively connected to wells containing A. caccae FIG. 11, bottom).

### Example 5—Operation of Splitting SlipChip without Agarose

A SlipChip device containing 1,000 compartments was designed and fabricated. Each compartment is composed of one well on the first plate and a duplicate well on the second (opposing) plate. During cultivation, the two wells were combined to allow growth of colonies inside a single compartment. The two plates were then separated and each colony was split in two, creating an identical copy of each colony array on either side of the split chip so that one copy can be used for destructive assays that require cell lysis, and the other copy can be used to preserve the live micro-organisms.

To facilitate visualization of this technical process, operation of the cultivation-SlipChip was illustrated with a red dye experiment (FIG. 12). For clarity, the following narrative both describes what happens to cells and colonies during the operation of the SlipChip, and also points out the corresponding images of the red dye experiments. The device was designed so that wells on one side of SlipChip overlapped with channels on the other plate and each plate contained both wells and channels (FIG. 12A). First, the suspension containing cells of interest was loaded into the channels and wells. This loading is shown as the loading of red dye in FIG. 12B. Then, the loading channels and wells were separated by slipping, and single bacterial cells were stochastically confined in wells. Duplicate wells on either side of the chip were combined as one compartment. This step is shown as the formation of fluid volumes of red dye solution (FIG. 12C). The sample in the loading channel was removed by purging with a vacuum so that air could fill the channel to support bacterial growth (FIG. 12D). It was observed that air could be introduced into channels and that the aqueous solutions (e.g., of red dye) remained in the wells and were not removed by the vacuum. The device was then incubated to grow bacterial colonies. To minimize loss of oil and water during incubation, the device was placed in a Petri dish saturated with the vapor of oil and water. Prior to subsequent splitting, oil was loaded into the device channels to replace air (FIG. 12E). The two plates were then slipped apart to separate the two wells that made up each compartment (FIG. 12F). The chips were designed so at this position, the through-holes on the top and bottom plates came into alignment so the device could be placed onto a holder for controlled splitting.

Air was introduced into the loading channel by removing the fluid with a vacuum. Since the SlipChip is not bonded, the gap between the two halves can serve as an oil reservoir, and it was observed that oil could flow back to the channel when the vacuum was released. In order to maintain the air supply and prevent oil from flowing back into the channel, repeated purging was conducted, with purging three to five times before loading the sample (FIG. 12A) being sufficient to prevent oil from returning during cultivation.

During the process of separation, fluid volumes can be partially released from the structure. At the same time, immiscible oil that is used for preventing evaporation of fluid volumes can flow into the gap of the chip and cause merging and cross-contamination of fluid volumes. In addition, the top and bottom plates of SlipChip might be shifted and misaligned. To address these issues, a holder was designed (FIG. 1 and FIG. 2A) to keep the top and bottom plate from shifting horizontally during separation. The holder was fabricated by standard machining Three pins for aligning SlipChips were inserted into the holder. Two glass spacers between the holder and the top plate were made by cutting 1 mm thick microscope slides into small pieces and glued to the holder on the edges with 5-min epoxy. The edge of the bottom plate was also removed to fit into the bottom part the holder with the glass spacer. The center part of the holder was cut away to facilitate imaging. Through holes on SlipChip were fabricated to align the SlipChip to the holder. Markers to define positions for through holes were incorporated into the design of photomasks and then transferred to the device by photolithography and wet-etching. To fabricate through holes, the device was first aligned to the laser stage using the etched marker, then ablated by laser machining (Resonetics RapidX250 system) with constant energy mode of 100 mJ with repetition rate of 80 Hz using 75-mm lens. The SlipChip was separated under a bath of tetradecane oil to prevent evaporation and separation was achieved by gravity.

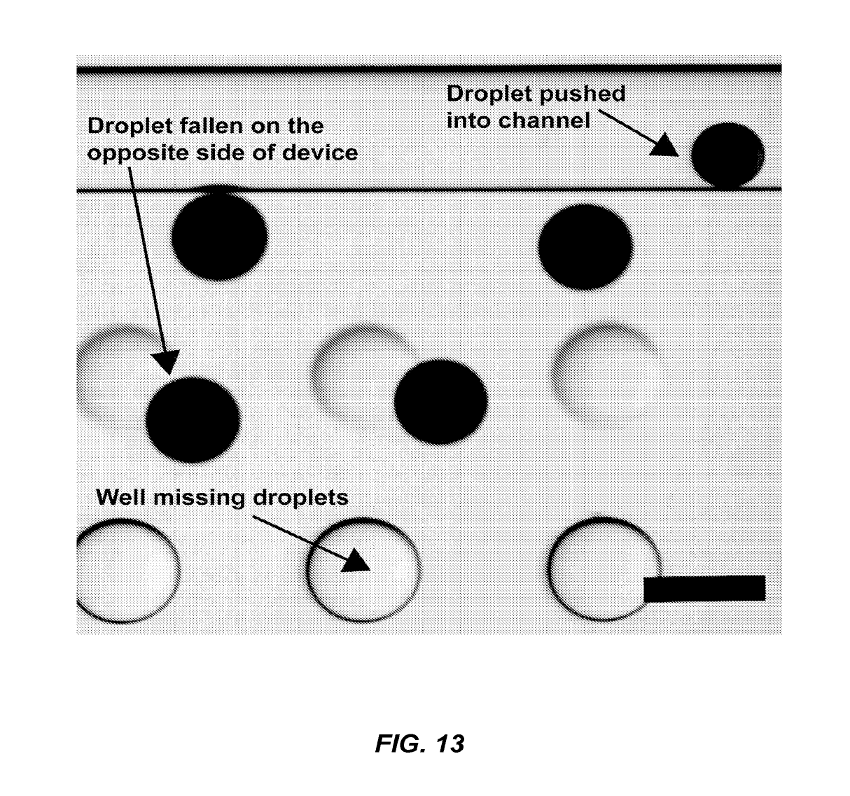

During splitting, oil entered the SlipChip through the gap between the two glass plates. The micro-structures on SlipChip could no longer hold the position of each fluid volume due to this oil flow. Although the holder could keep the two pieces of glass plates well aligned, the aqueous fluid volumes in the micro-wells may not stay in the same positions as the ones before splitting (FIG. 13). Fluid volumes may either be pushed out of the micro-wells by oil flow, or stuck on the opposite side of the device.

### Example 6—Operation of Splitting SlipChip with Agarose

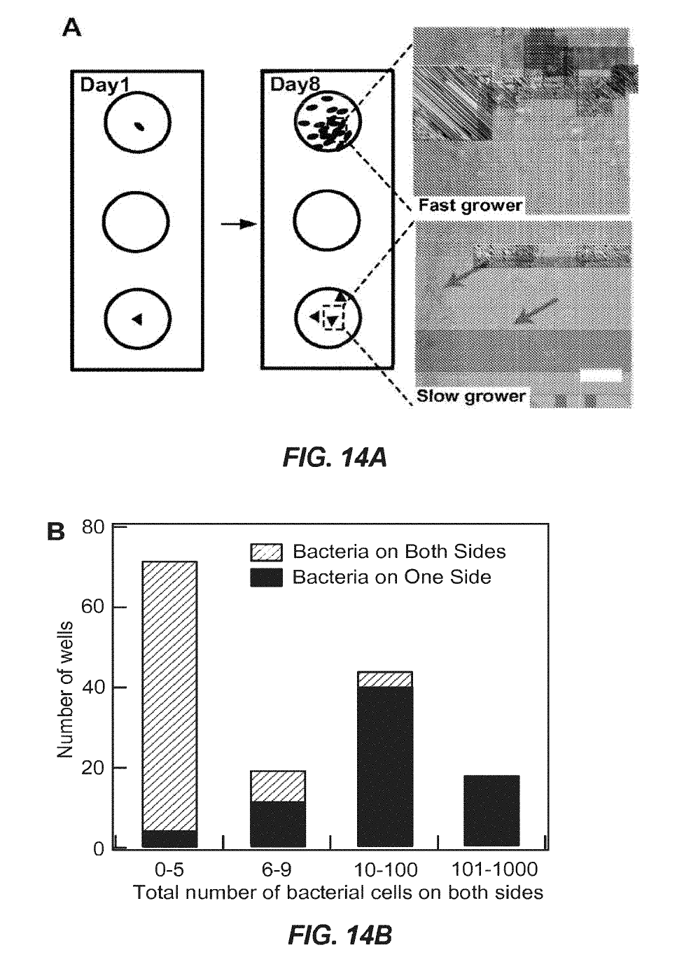

To hold the fluid volumes in the microwells and minimize the effect of shear from oil flow, an ultra-low gelling temperature agarose was added to increase the viscosity of the fluid volume in a SlipChip system as described in Example 5. The addition of agarose did not inhibit bacterial growth or generation of duplicate copies, as shown in FIG. 14B. To test if this setup could keep the fluid volumes in microwells during splitting, 1% agarose aqueous solution was loaded onto SlipChips while warm (˜37° C.). The device was then incubated on a 10° C. chilling plate to gellify the agarose while remaining above the melting point of tetradecane (8° C.). The splitting then took place within 3 minutes after SlipChip was placed on the holder. The shape of fluid volumes changed during splitting, indicating the fluid volume was partially released from the micro-structure (FIGS. 2B and C). This shape change was not due to evaporation of fluid volumes because the fluid volume shape could be restored by clamping the two plates back together. 2,000 wells on the whole device were analyzed with a stereoscope (FIG. 2D shows a part of the device) and no missing fluid volumes or cross-contamination among wells during splitting was observed. The above was then conducted with the concentration of agarose varying from 0.3 to 2%. While 1% agarose was used for some preliminary experiments, 0.5% was found to be the minimum concentration that gave reliable results.

### Example 7—Cultivation of Clinical Biopsy Sample

A SlipChip device was prepared in an anaerobic chamber using AM2 medium supplemented with 0.5% ultra-low gelling temperature agarose and loaded with a diverse bacterial community of anaerobes using microbes from a microbial suspension obtained from a mucosal biopsy from the colon of a healthy human volunteer. The devices were then incubated at 37° C. in an anaerobic chamber for 8 days. Afterwards, devices were imaged with a microscope to visualize growing microbial colonies. Bacteria from a microbial suspension obtained from a mucosal biopsy from the colon of a healthy human volunteer were observed to grow on the SlipChip. Also, fast- and slow-growing bacteria in a clinical sample were successfully separated on SlipChip (FIG. 14A) Additionally, slipping successfully generated two daughter colonies if, after growth, the original single cell gives rise to a colony consisting of more than 10 cells (FIG. 14B).

### Example 8—on Chip Dilution

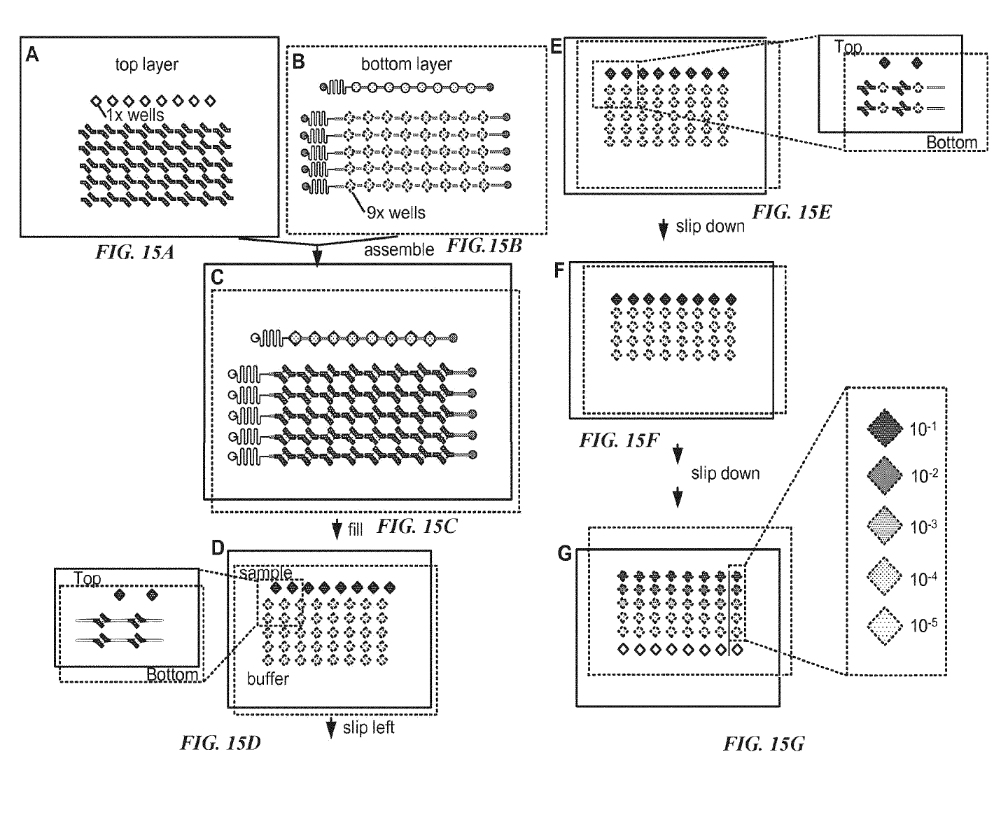

A SlipChip was designed and fabricated to perform 5 serial dilution steps in parallel FIG. 15). It comprises two components: a row of shallow wells that contains sample and an array of deep wells that are filled with buffer solutions for dilution. Using the SlipChip to perform serial dilutions involves three general steps: (a) load buffers, (b) load samples, and (c) multi-step slip to dilute. After filling the SlipChip by pipetting, the two plates of the chip are slipped to separate ducts from wells. As the ducts are separated from the wells, they are also moved out of the slipping path (FIG. 15D and FIG. 17e insets). The wells containing sample are brought into contact with the wells containing buffer, and the sample is diluted. The mixing ratio, or dilution factor, is determined by the ratio of well sizes. Further steps of slipping operate by the same principle and thus serial dilutions are performed.

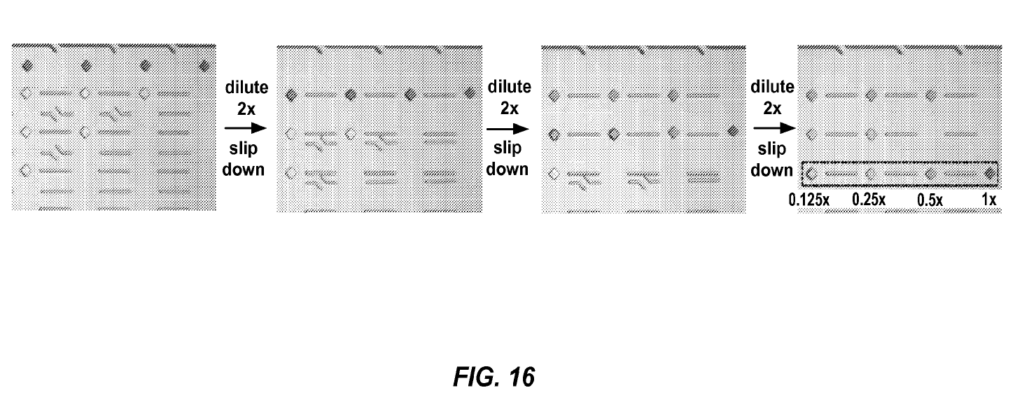

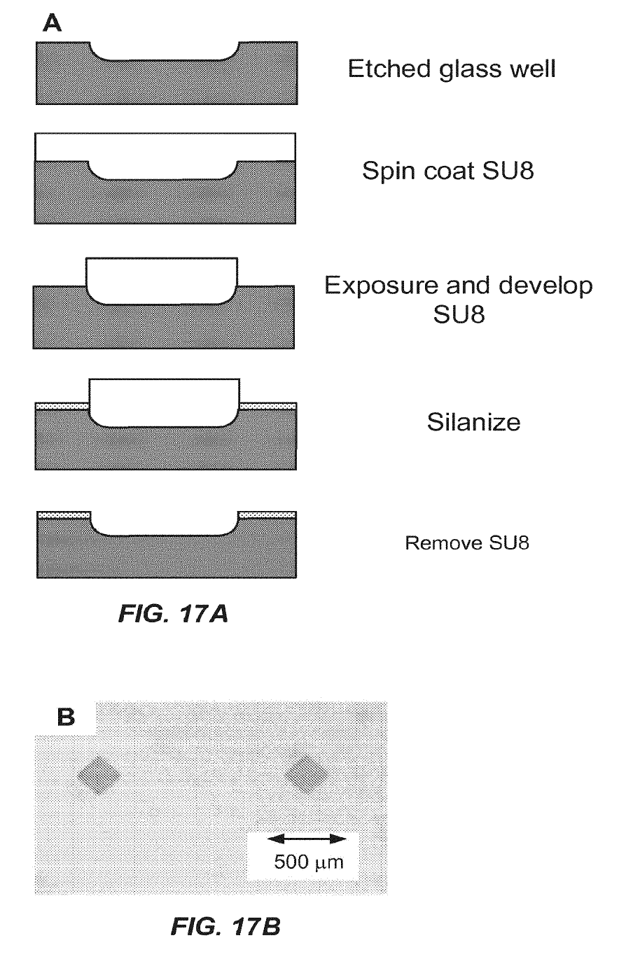

The SlipChip is composed of two layers of microfabricated glass: The top layer contains all the inlets and outlets, ducts for the sample, and wells for the buffer solution. This device was designed to perform a serial of 2-fold dilution. All wells and ducts are etched 60 μm deep. The top and bottom wells are the same dimensions, so 2-fold dilution was obtained after each step of slipping and mixing. To visualize the operation of the serial dilution device, an aqueous solution of red dye (0.1 M Fe(SCN)₃) was loaded onto SlipChip. Millipore water was used as the dilutant. After 3 slipping steps, a 2³-fold dilution was achieved, as can be seen from the color change in FIG. 16. Only 2³-fold dilution is shown, as the color of the Fe(SCN)₃ solution faded after a 10-fold dilution.

### Example 9—On Chip Logarithmic Dilution

SlipChip devices, such as those in Example 8, were designed to perform logarithmic serial dilutions across a wide dynamic range. All wells are 76 μm deep and ducts are 30 μm deep. The top layer contains all the inlets and outlets, ducts for the sample, and wells for the buffer solution. The bottom layer contains 10 μm shallow wells for the sample and 30 μm deep ducts for the buffer solution. The surfaces of the device were silanized to be hydrophobic while keeping the 10 μm deep wells hydrophilic. 10 μm shallow wells were used to decrease diffusion time in and out of the well as well as to minimize the volume for the diluent wells. To make hydrophilic wells, the glass plate was piranha cleaned (1 part 30% hydrogen peroxide, 3 parts sulfuric acid by volume), washed twice with Millipore water, and then dehydrated on a 220° C. hot plate for more than 2 hours. The plate was cooled down to room temperature and spin-coated with a 20 μm thick layer of SU8 3010 (FIG. 17A). The plate was next aligned and covered with a photomask that protected the areas on the plate that were to be hydrophobic, so that only the SU8 in the wells remained after developing. The SU8 in the wells was used to protect the wells and prevented them from being made hydrophobic. Finally, the glass was dried by baking in 120° C. for 15 minutes. The glass plates were cleaned and subjected to an air plasma treatment at 300 mTorr for 5 minutes, and then the surfaces were rendered hydrophobic by silanization in a vacuum dessicator for 5 hours with Tridecafluoro-1,1,2,2-tetrahydrooctyl-1-trichlorosilane. After silanization, the glass plates were rinsed three times with 20 ml anhydrous toluene, three times with 30 ml anhydrous ethanol, three times with 30 ml ethanol/H₂O (50%: 50%, v:v), and three times with 30 ml Millipore water. The plates were baked in a 120° C. oven for 15 minutes. Finally, the SU8 in the wells was stripped by immersing the glass plates in Piranha for less than 1 min. The plates were then washed twice with Millipore water and dried 120° C. for 15 minutes. The well is made hydrophilic to control the shape and the volume of the aqueous fluid volume within the hydrophilic well, and also to prevent de-wetting from the shallow well (FIG. 17B).

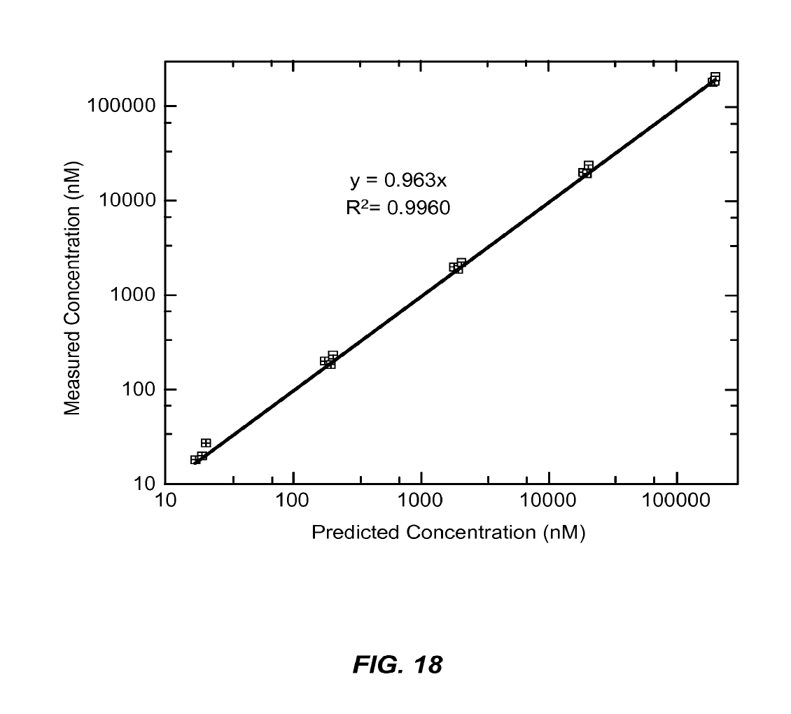

A solution of fluorescent dye was used to quantify the success of logarithmic serial dilution across a wide dynamic range on this SlipChip. Alexa Fluor 488 hydrazide (2 mM, Invitrogen) in PBS buffer (lx, pH 7.4) was loaded by pippetting into the sample channel. 1×PBS buffer solution was loaded into the buffer channel. The SlipChip was slipped under a Leica MZ 16 stereoscope to form isolated fluid volumes first. Then, the sample wells were combined with the buffer wells sequentially. After each slipping step, there was a wait step of 10 minutes to allow for the diffusion of the fluorescent dye. After 5 steps of slipping, the device was quickly transferred to a Leica DMI6000 microscope (Leica Microsystems) with a 20×0.7NA Leica objective and a Hamamatsu ORCAER camera. Different settings were used for low concentrations and high concentrations of Alexa Fluor 488 solutions in order to obtain fluorescent images across a high dynamic range. A L5 filter with an exposure time of 30 ms and 100% lamp intensity was used to collect Alexa Fluor 488 fluorescence from 20 nM to 2000 nM. A L5 filter with an exposure time of 2 ms and 30% lamp intensity was used to collect Alexa Fluor 488 fluorescence from 20 μM to 200 μM. Images were acquired and analyzed by using Metamorph imaging system version 6.3r1 (Universal Imaging). The concentrations from on-chip dilutions were calculated from the fluorescence intensities. To calibrate the microscope, the fluorescent intensity of a fluorescence reference slide for the L5 filter was recorded and used for background correction. 20 nM, 50 nM, 200 nM, 500 nM, and 2000 nM Alexa Fluor 488 hydrazide solutions in PBS buffer were used to obtain a calibration curve to determine the concentration of fluorescent dyes on the lower end of the concentration range. 10 μM, 20 μM, 50 μM, 100 μM and 200 μM Alexa Fluor 488 hydrazide solutions in PBS buffer were used to obtain another calibration curve to determine the concentration of fluorescent dyes on the higher end of the concentration range. Well depth was measured with a Veeco Dektak 150 profilometer and the volume of the wells was calculated with the assumption that etching is isotropic. After 5 slipping steps, a 10⁵-fold dilution was achieved with good agreement between experimental results and theoretical calculation (FIG. 18).

### Example 10—Retrieval of Fluid Volumes

After a SlipChip device, such as those described in previous examples was split, one copy of the device was mounted onto an alignment holder (e.g. FIG. 1, FIG. 2A). 1 μL of an aqueous buffer solution was aspirated with an Eppendorf pipettor. The spacing between wells on the SlipChip was designed to be larger than the outer radius of the pipette tip to prevent cross-contamination between wells. This buffer solution volume merged spontaneously with the 2 nL fluid volume on the SlipChip when brought into contact to minimize the total interfacial area FIG. 3). The combined fluid volume could then be aspirated back into the pipette tip and used for spreading plates or testing with PCR and subsequent sequencing. This method can be modified to accommodate situations such as wells with higher density or when pipetting cannot be controlled accurately. In this case, the fluid volumes in neighboring wells can first be removed by this method, which gives more space for subsequent handling of the target fluid volume.

### Example 11—PCR Identification of Target Colonies

E. coli cells were enriched with 50 μg mL⁻¹ of Ampicillin in LB at 34° C. overnight (12 hours) in a rotary shaking incubator at 200 rpm to reach stationary phase. To synchronize cells, overnight culture of each species was then diluted 100-fold and cultured with 10 μg mL⁻¹ of Ampicillin and 40 μmol L⁻¹ IPTG in LB media for 3 hrs. Cells were then pelleted at 3000×g for 5 min and washed 5 times with 1 mL of ice-cold 1×PBS buffer. Cells were finally suspended in 10 μg mL⁻¹ of Ampicillin and 40 μmol L⁻¹ IPTG in LB media and cell suspension was serially diluted with 10 μg mL-1 of Ampicillin and 40 μmol L⁻¹ IPTG with 0.5% of ultra-low gelling temperature agarose in LB media and mixed to a final density of 2×104 and 2×103 CFU mL-1 for E. coli strains with GFP and DsRed genes, respectively, and loaded onto a SlipChip device. Individual cells were compartmentalized and the SlipChip was incubated at 34° C. for 3 hours for cultivation and then split into daughter chips (FIG. 5A).

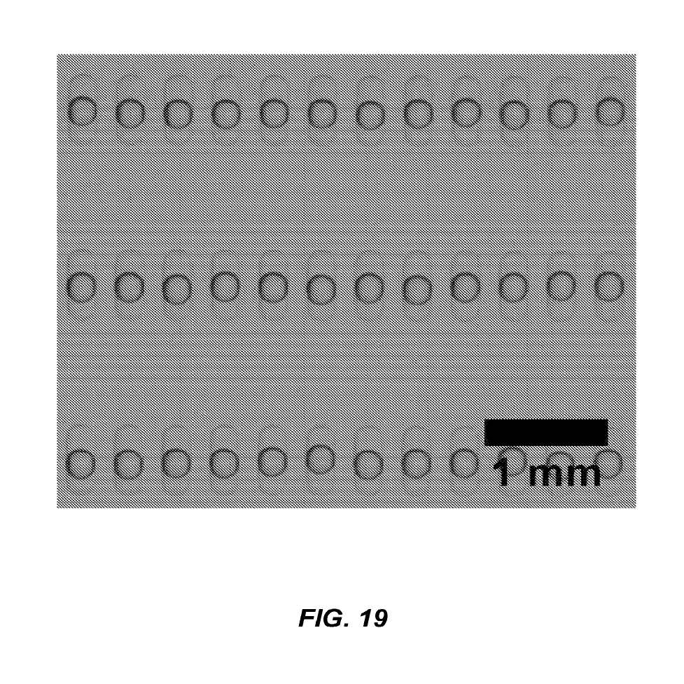

One chip was mixed with PCR reagents (FIG. 19) containing primers targeting the plasmid of DsRed (FIG. 5B), and the other was imaged with a fluorescence microscope to check for the presence or absence of fluorescent proteins (FIG. 5C). Fluorescence images were acquired with a Leica DMI6000 microscope (Leica Microsystems) with a 10×/0.4NA Leica objective and a Hamamatsu ORCA-ER camera with 1× coupler. An L5 filter with an exposure time of 500 ms was used to collect images. For quantitative analysis, fluorescent intensity of a fluorescence reference slide for L5 filter was recorded and used for background correction. Images were acquired and analyzed by using Metamorph imaging system version 6.3r1 (Universal Imaging) and ImageJ by the National Institutes of Health. Fluid volumes containing E. coli expressing DsRed were PCR positive compared to blank wells that contained no bacteria and to wells containing GFP-labeled E. coli FIG. 5B), indicating that only the targeted genes were amplified. To confirm that the PCR positive wells were indeed from the plasmid of DsRed, the expressed fluorescent proteins were monitored using fluorescence microscopy. 125 wells were observed that contained colonies with GFP, and 12 wells were observed containing red fluorescent E. coli, which matched the PCR results (FIGS. 5D and 5E). One well was observed that showed increased fluorescence intensity in the PCR result, but no bacterial colony was detected in the other copy, which indicates that the well can have contained non-growing cells or free DNA from the solution.

### Example 12—Isolation of B. vulgatus from a Clinical Sample

Cell scrapers were used to collect cultivar of a frozen microbial suspension obtained from a mucosal biopsy from the colon of a healthy human volunteer on the Wilkins-Chalgren anaerobe (WCA) plate. DNA was purified from pooled cell using QiaAmp DNA Mini kit. 50 ng of DNA and B. vulgatus-specific primers were used for PCR. Positive PCR product was validated by Sanger sequencing and aligned to the sequence of B. vulgatus from GenBank.

A SlipChip device as described previously was loaded with the appropriate dilution of the sample with WCA medium with 0.5% ultra-low gelling temperature agarose. Incubation on the SlipChip was as short as overnight (8 hours) for isolating B. vulgatus, as the SlipChip had the capability to perform PCR assays with only 10-100 cells (FIG. 5E). This is compared with the need for thousands to millions of cells to form a colony on an agar plate, where it normally takes three to five days of incubation on agar plates to obtain enough biomass for making stocks and running assays with colonies. The SlipChip device was split and PCR was performed on samples from one plate to identify positive colonies. Five colonies were selected from the duplicate wells for PCR positive wells, and three of them could be scaled up on an agar plate. Those two false positive results can come from lysed or non-growing cells, as this example was performed with a frozen sample and the viability of microbes is compromised during the freeze-thaw cycle. Sequencing was used to confirm that those isolates were indeed B. vulgatus.

### Example 13—Plate Wash on SlipChip “Chip Wash”)

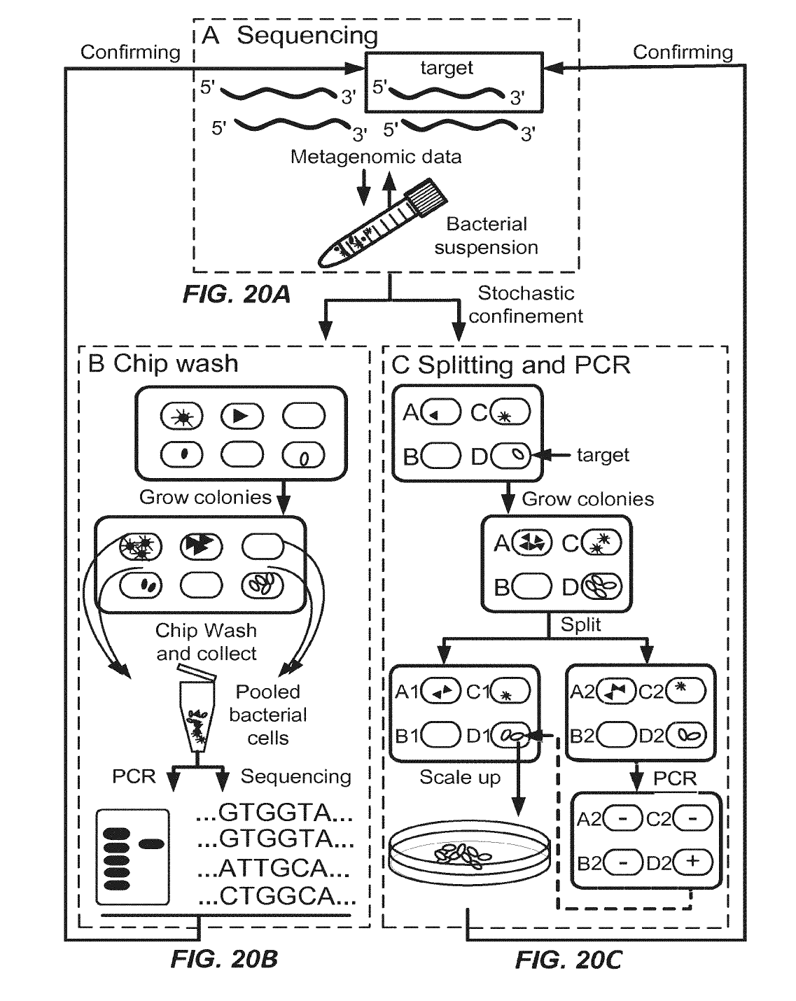

Single bacterial cells from clinical samples are stochastically confined and cultivated on a SlipChip device as described previously. The microcolonies are then collected in a single tube by flushing the microwells for DNA extraction. The composition of the cultivar grown on SlipChip can be analyzed by either sequencing or target-specific primers in order to determine whether the cultivation conditions for that chip allowed the growth of the target microorganism. This chip wash method can be repeated until the optimal growth condition for the target is found (e.g. FIG. 20).

### Example 14—Plate Wash on SlipChip “Chip Wash”) with E. coli

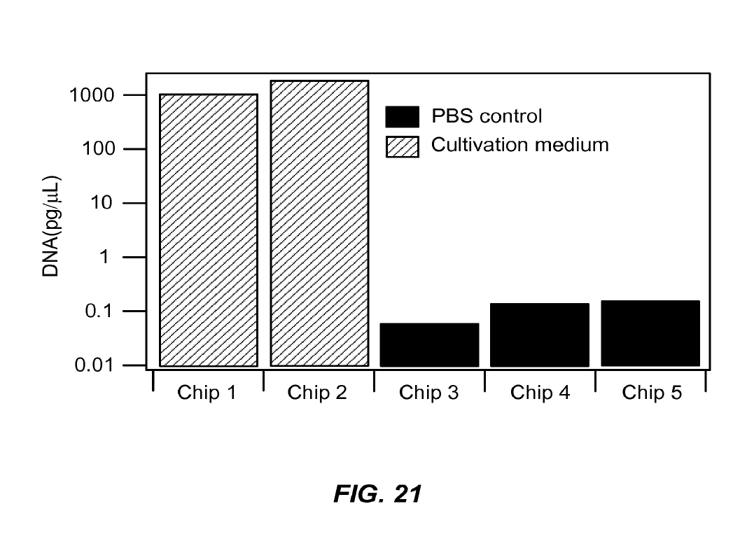

E. coli cells labeled with DsRed fluorescent proteins were enriched with 50 μg mL⁻¹ of Ampicillin in LB at 37° C. overnight (12 hours) in a rotary shaking incubator at 200 rpm. Overnight culture was then diluted 100-fold and cultured with 10 μg mL⁻¹ of Ampicillin and 40 μmol L⁻¹ IPTG in LB media for 3.5 hrs. Cells were then pelleted at 3000×g for 5 min and washed 3 times with 1 mL of 1×PBS buffer. Cells were serially diluted to a final density of 10⁵ in 10 μg mL⁻¹ of Ampicillin and 40 μmol L⁻¹ IPTG in LB media or PBS buffer, which does not support growth of bacteria, as a negative control and loaded onto a SlipChip device as described previously. The SlipChip was incubated at 37° C. overnight. Genomic DNA was purified from chip wash solutions using a QiaAmp microkit according to the manufacturer's protocol. For calibration, genomic DNA was purified from macroscopic liquid culture, quantified by a Quanti-it DNA high sensitivity quantification kit, and serially diluted in AE buffer containing 0.01 mg mL⁻¹ of BSA. Quantitative PCR (qPCR) was performed with 27F and 534R primers. A 10,000-fold increase of DNA concentration was observed (FIG. 21), suggesting that for this particular model system, non-growing cells contribute to 0.01% of the genetic material recovered from chip wash.

### Example 15—Plate Wash on SlipChip “Chip Wash”) with Two Species Model Community

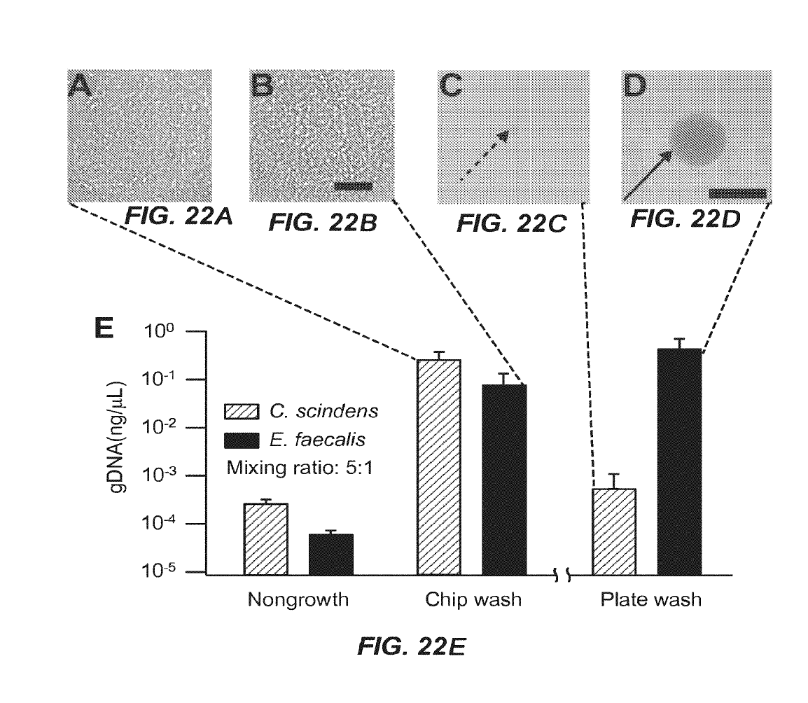

A mixture of Clostridium scindens and Enterococcus faecalis was cultivated at a 5:1 ratio on a SlipChip device and on agar plates. The genomic DNA of the starting inoculum and chip wash solution were extracted and quantified by qPCR. Cultivation on the chip followed by chip wash resulted in a ˜1,000-fold increase of DNA for each strain compared to DNA from the starting inoculum used as a non-growth control (FIG. 22E), showing that chip wash can be used to detect microbial growth.

E. faecalis grew faster than C. scindens on agar plates, as observed from the difference in colony size on day 1 (FIG. 22C D). The cultivation medium has a similar carrying capacity for the two strains. The two strains grew on the chip to a comparable density on day 1 (FIG. 22A B). As shown by the quantity of genomic DNA recovered from the two strains, sampling on day 1 by plate wash resulted in a ˜1,000-fold bias toward rapidly growing bacteria, while the chip wash method effectively corrected this bias, as the genomic DNA was comparable for each strain (FIG. 22E).

### Example 16—Plate Wash on SlipChip “Chip Wash”) with Four Species Model Community

A model community was selected consisting of four members: Anaerostipes caccae, Bifidobacterium infantis, Clostridium scindens, and Enterococcus faecalis. This community represents the two dominant phyla of the adult human distal gut microbiota: Firmicutes and Bacteroidetes. Schaedler Anaerobe Broth medium was chosen to support growth of all four species. To exclude the possibility that SlipChip does not support growth of the four species, the four species were loaded separately in different devices. After one day of cultivation on SlipChip, all four species grew to dense micro-colonies.

Next, Anaerostipes caccae, Bifidobacterium infantis, Clostridium scindens, and Enterococcus faecalis were enriched in Schaedler Anaerobe Broth medium overnight and then cultured for eight hours to synchronize the cells to mid-exponential phase. Cells were evenly mixed and diluted to 10⁵ CFU mL⁻¹ in Schaedler Anaerobe Broth medium and loaded onto SlipChip. The SlipChip was incubated for 24 hours and analyzed using the chip wash method as in Example 13 or 14. Genomic DNA was purified using a QiaAmp microkit according to the manufacturer's protocol. Genomic DNA was purified from macroscopic liquid culture, quantified by a Quanti-it DNA high sensitivity quantification kit, and serially diluted for calibration. By using the starting inoculum as a non-growth control, approximately 1,000-fold amplification from growth was observed. While the plate wash was strongly biased towards Enterococcus faecalis when sampled on day 1, no significant bias between Clostridium scindens and Enterococcus faecalis was observed with chip wash (FIG. 22B). This demonstrates that the chip wash method can reliably detect species grown on a SlipChip.

### Example 17—Identification of Growth Conditions for OTU158

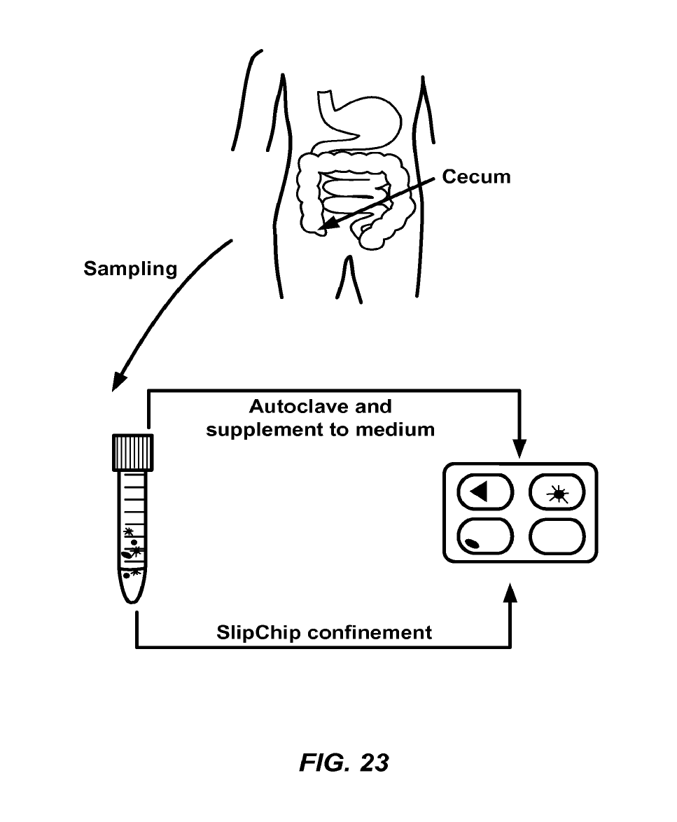

Samples were collected from the human cecum (FIG. 23) by two methods: brush mucosal biopsies were obtained by a brushing technique and wash fluid was obtained by a lavage technique. The wash fluid was autoclaved and spiked into the cultivation medium M2LC. The brush sample was loaded onto SlipChip at 10⁵ CFU mL⁻¹ and allowed to incubate for 3 days to grow colonies. The cultivar was then collected using the chip wash method described previously, as in Examples 13-15. The same amount of inoculum was also cultivated on agar plates for comparison. Rumen fluid was spiked into the medium M2GSC.

A 16S high-throughput sequencing survey of the cultivar was conducted with two regions: V4 and V1V3. The OTU table from chip wash and plate wash was sub-sampled to 12599 reads per sample and summarized at family level. Cultivar grown from SlipChip devices were different from cultivar grown on plates in both composition and relative abundance of microbes. For example, more Clostridium XIII and Bifidobacterium can be observed from the plate wash, while some low abundance members such as Gordonibacter, Anaerostipes, Oscillibacter and Silanimonas can only be observed from the chip wash method. This discrepancy likely results from the difference between bacterial growth kinetics on agar plates and on chip, as well as the differing cultivation conditions (the luminal fluid was used for on-chip cultivation, while ruminal fluid was used for agar plates). Reads classified as Oscillibacter were able to be assigned to OTU_158_V1V3. PCR with species-specific primers for OTU158 was performed, and results were validated by Sanger sequencing, which confirmed that OTU158 can be found from the cultivar.

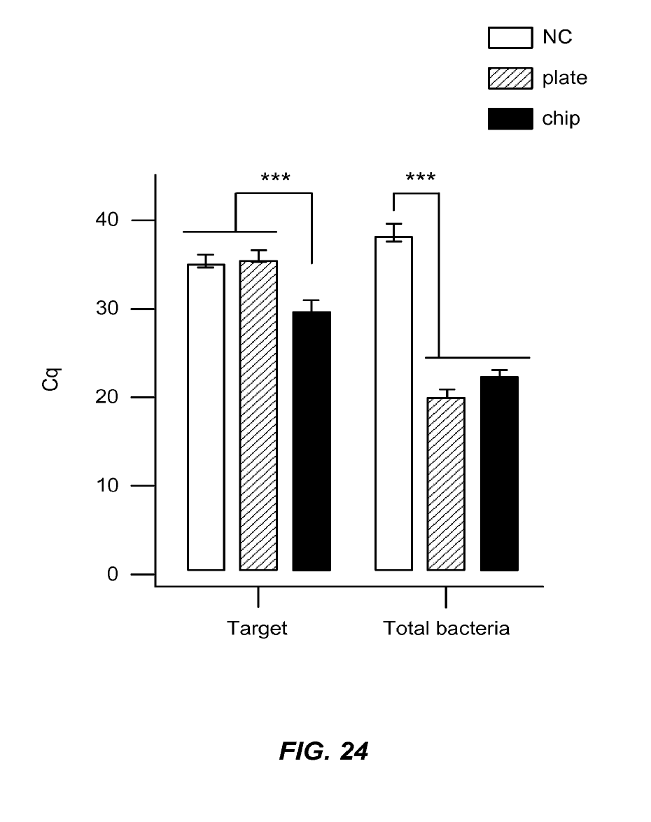

Results of 16S V4 high-throughput sequencing were further validated by qPCR (FIG. 24). The null hypothesis was that in blank water, plate wash from medium M2GSC, and chip wash from medium M2LC, the concentrations of genomic DNA from OTU158 were identical. The concentration of genomic DNA from OTU158 was higher than that of both the blank negative control and the cultivar from plate wash. To test if both plate wash and chip wash contained bacterial DNA, qPCR was performed with 16S V4 universal primers. Both plate wash and chip wash solutions had DNA concentrations higher than the blank negative control, and the plate wash solution had a slightly lower Cq value.

To test if luminal fluid is required for growth of OTU158, cultivar grown on M2GSC medium was obtained by chip wash and tested with qPCR. The difference between the cultivar and blank negative control was not statistically significant, and indicating that chip wash with M2GSC medium was not able to recover OTU158. It was determined that M2LC is a suitable cultivation medium to grow OTU158.

### Example 18—Isolation of “Candidatus Caecococcus Microfluidicus”

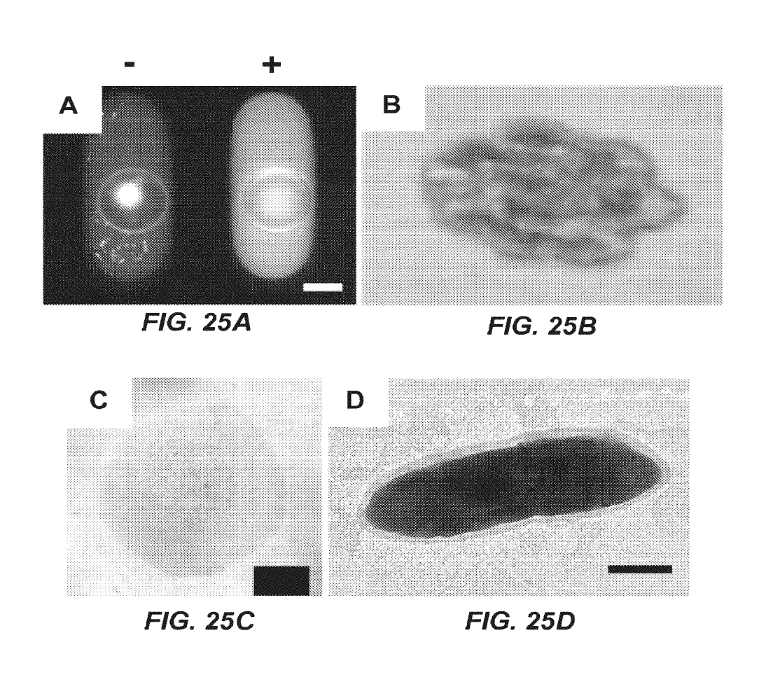

Cultivation was performed with the M2LC medium as prepared in Example 16, loaded onto a SlipChip device as described previously. After cultivation, the two SlipChip plates were separated as described previously and colony PCR was carried out on each micro-colony with primers targeting the OTU_158_V1V3 (OTU158) region of interest. Two PCR positive hits were observed (one of them shown in FIG. 25A) from a single device loaded with ˜500 microbial colonies. An image of PCR negative well next to the hit is shown in FIG. 25A on the left. Although cell material was stained by SYBR Green and showed fluorescence, the solution phase was clearly PCR negative. One of the positive wells was scaled-up on the M2GSC agar, and the photograph of an intact “scale-up” culture after three days of incubation is presented in FIG. 25B. The culture contained multiple cells, as shown, due to the presence of multiple seeds transferred from the SlipChip. Colony PCR was performed on this isolate with both species-specific and universal primers, which confirmed the presence of OTU158.

Plates were repeatedly streaked with single colonies (e.g. FIG. 25C) of target cells for purification. After five times of streaking the plates, 16s rRNA gene was used to determine the purity of the culture. Genomic DNA for PCR amplification was isolated using QiaAmp kit following the manufacturer's protocol with the following modification: bead-beating step was added, using lysing matrix B (MP Biomedicals 6911-500) that was shaken using a Mini-Beadbeater-16 (BioSpec Products, Inc.) for 1 minute. The 16s rRNA gene was amplified by PCR using AccuPrimer Pfx DNA polymerase (Invitrogen). Primers 27F and 1492R were used for PCR amplification. PCR amplification was performed by a Biorad thermocycler with 2 minute incubation at 95° C., followed by 34 cycles at 95° C. for 15 seconds, 55° C. for 30 seconds, and 68° C. for 90 seconds. Amplified PCR product was cloned into TOPO vector and transformed into TOPO10 E. coli cells on LB/Amp+ medium. The plates were incubated at 37° C. overnight and single colonies were picked for liquid culture. Plasmids were purified from cells using Qiagen Miniprep kit. Plasmid DNA was then amplified by PCR with the same protocol as described above. PCR products were purified using QIAquick PCR purification kit and sequenced by Laragen.

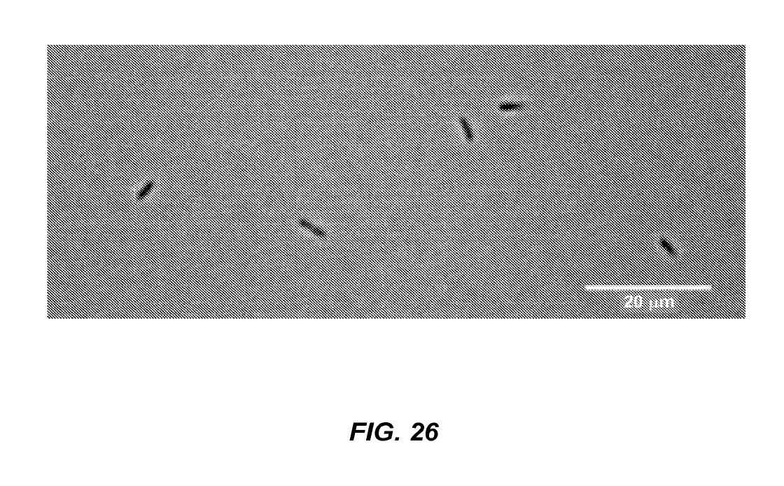

TEM was performed with 200 mesh formvar/carbon grids on TECNAI 120 keV TEM (FEI, Hillsboro, Oreg.) equipped with a Gatan 2k by 2k CCD camera for image acquisition (FIG. 25D). Optical microscopy of the isolate was obtained by suspending the cells in PBS buffer and imaged using a 63×1.2 NA Leica objective with a Leica DMI6000 microscope (Leica Microsystems) and a Hamamatsu ORCAER camera. Rod-shaped bacterial cells were observed from both the TEM image (FIG. 25D) and optical microscopy (FIG. 26).

Two 16S rRNA gene sequence types were obtained from the culture. They are 99.4% identical to each other and affiliated to the family Ruminococcaceae, previously referred to as Clostridium cluster IV. 16S rRNA targeted FISH was carried out following established protocols. In brief, formaldehyde- and ethanol-fixed samples were hybridized at 46° C. with FAM- and Cy3-labeled oligonucleotide probes for 16 hours in a formamide-containing humid chamber. To test whether cell wall digestion leads to an increase in fluorescence detection and/or labeling intensity, before hybridization, samples were pre-treated with either (i) 10 mg mL⁻¹ lysozyme in TE buffer (1 h at 37° C. in a humid chamber); (ii) 15 μg mL⁻¹ proteinase K in TE buffer (10 min at room temperature, i.e. 23° C.) followed by soaking in 0.01 M HCl (10 min at 23° C.); or (iii) a 1:1 mix of acetone:methanol (15 min at 23° C.). Formamide concentrations in the hybridization buffer were as recommended: 20-35% for probe mix EUB338 I-III and control probe NonEUB338; 35% for probe Arch915; 20% for probe EUK516. The two newly designed probes Clostr183-I and Clostr183-II were hybridized at 15% (at concentrations >20% no fluorescence signal was observed). Via competition for the same binding site, these probes are able to distinguish between the two 16S rRNA gene sequence types obtained from our culture. After hybridization, slides were washed for 10 min in pre-warmed washing buffer at 48° C. Then, they were dipped into pre-cooled deionized water (4° C.) and dried using pressurized air. Slides were mounted with DAPI/Citifluor and analyzed using an Olympus BX51 epifluorescence microscope. Fluorescence images were analyzed using the software provided by the microscope manufacturer and ImageJ. No unspecific labeling was observed when control probe NonEUB338 was applied to the samples.

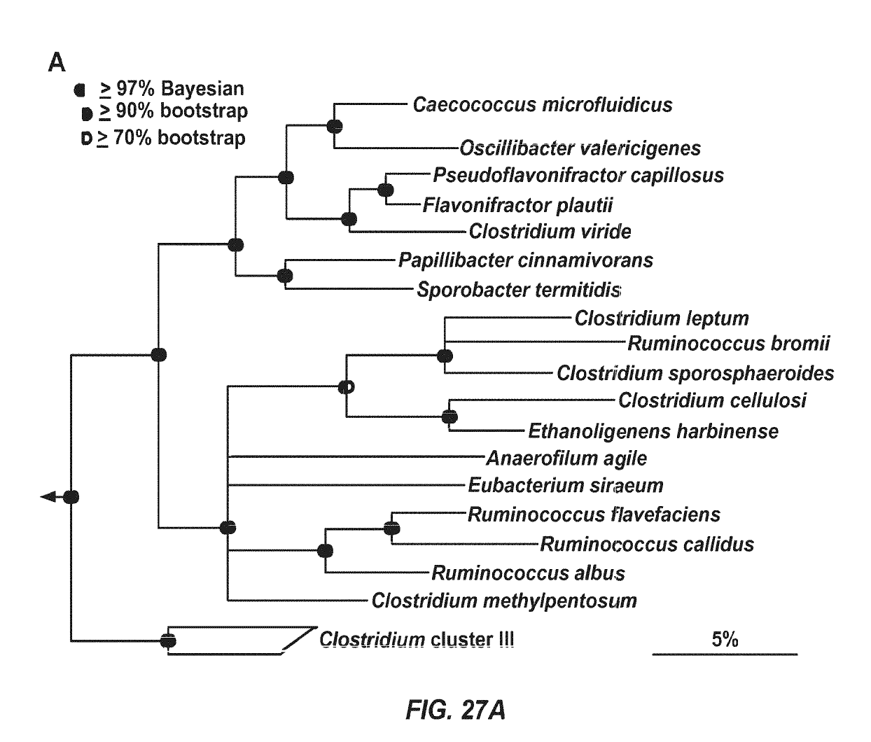

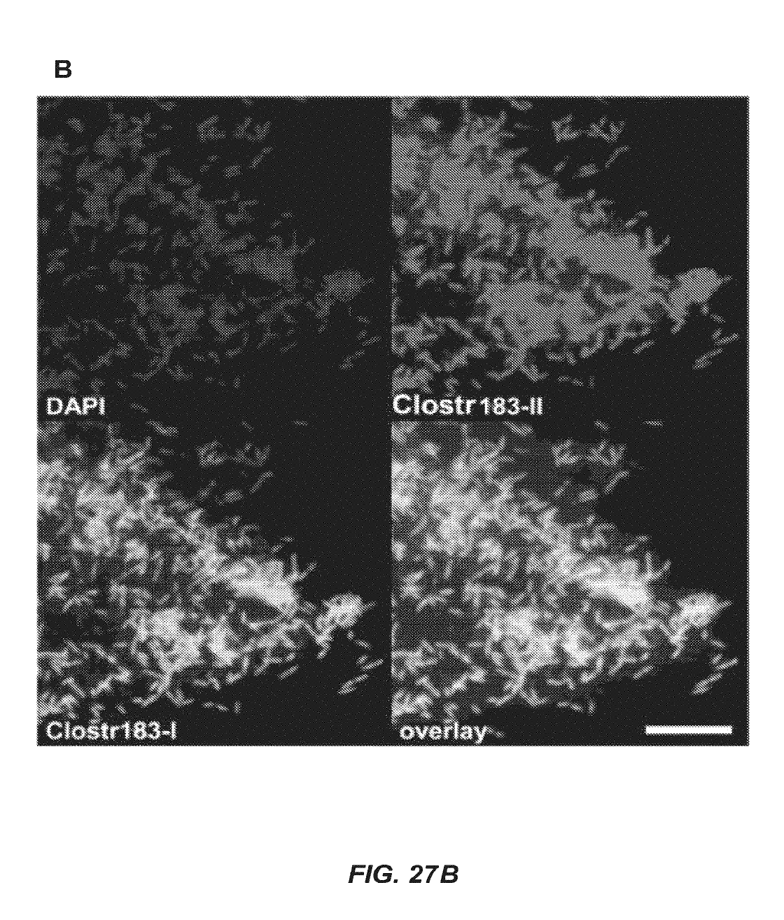

All FISH-positive cells bound both sequence type-specific FISH-probes (Clost183-SI and Clost183-SII, FIG. 27), as well as the general probe mix EUB33 81-III which specifically detects most members of the Bacteria. While no archaeal or eukaryotic cells could be detected in the culture, some DAPI-stained cells could not be stained via. FISH. In order to rule out that this was due to a limited accessibility of the respective cells, different cell wall permeabilization treatments were tested. However, none led to successful FISH-staining of these cells. Therefore, the respective cells likely have a ribosome content that is below the detection limit of mono-FISH, which may be due to sporulation of the respective cells. This idea is supported by the finding that in an analysis of the culture in stationary phase, most cells cannot be visualized using FISH, even when permeabilization steps are conducted. These FISH results demonstrate the presence of a single Ruminococcaceae species in the culture.

### Example 19—Culture and Analysis of Gut Microbes

Single microbial cells from a human and/or mouse gut biopsy, are stochastically confined on a device and incubated to allow growth of colonies. Cell lines are loaded onto a SlipChip device and incubated to allow growth of colonies. The two plates of the device from bacterial culture are separated, and each compartmentalized fluid volume splits into two, creating identical copies of each individual colony on each of the opposing plates. A functional assay is performed on the first plate to by overlaying the glass plate containing bacterial cells with the glass plate containing mammalian cell culture to identify the compartments containing the colonies with the function of interest. Expression of genes is visualized by performing an assay such as, for example, a fluorescent, colorimetric, chemiluminescent, or mass spectrometry assay. Corresponding colonies are then be retrieved from the second plate for other purposes, such as a scale-up culture of isolates of interest.

### Example 20—Cultivation of Sulfate Reducing Bacteria

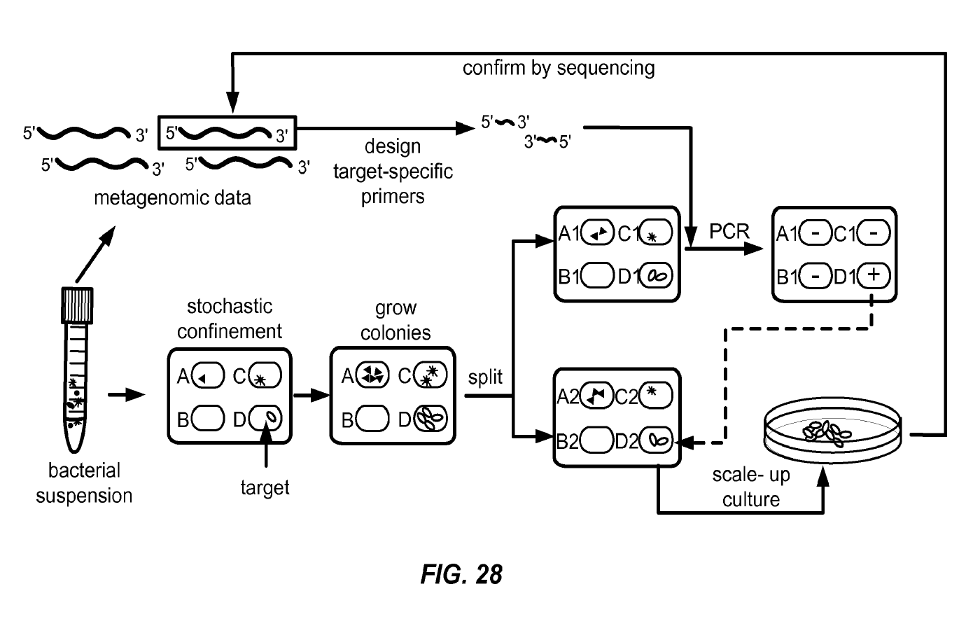

A sample suspected of containing sulfate-reducing bacteria is loaded onto a SlipChip device and stochastically confined. The device is incubated and colonies are grown. The two plates of the SlipChip device are separated, producing matching colonies in the paired wells from each plate. Samples from one plate are assayed for sulfate-reducing behavior, either by a genetic assay targeting relevant genes (e.g. FIG. 28), or by a functional assay for the presence of sulfate-reducing activity (e.g. by use of a fluorogenic substrate N,N-dibutyl phenylene diamine (DBPDA) to detect H₂S). Colonies found positive for sulfate-reducing are noted, and their matching colonies from the other SlipChip plate are cultivated for further study. A previously unknown sulfate-reducing bacterium is found in the sample.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-17. (canceled)

18. A method, comprising:
(a) providing a first substrate comprising a first defined volume;
(b) providing a second substrate comprising second defined volume, said second substrate coupled to said first substrate;
(c) loading a first sample into said first defined volume;
(d) bringing said first defined volume into fluidic contact with said second defined volume, wherein said first substrate remains coupled to said second substrate;
(e) mixing contents of said first defined volume with contents of said second defined volume, thereby producing a mixed sample;
(f) separating said first defined volume from said second defined volume, wherein a first part of said mixed sample is contained by said first defined volume and a second part of said mixed sample is contained by said second defined volume, wherein said first substrate remains coupled to said second substrate; and
(g) decoupling said first substrate from said second substrate, wherein said first part of said mixed sample remains contained by said first defined volume and said second part of said mixed sample remains contained by said second defined volume.

19. The method of claim 18, wherein said mixed sample further comprises a gelling agent.

20. The method of claim 18, wherein said mixing comprises diffusion.

21. The method of claim 18, wherein said mixing comprises sonication.

22. The method of claim 18, wherein said bringing said first defined volume into fluidic contact with said second defined volume is performed on an apparatus.

23.-35. (canceled)

36. A method, comprising:
(a) dispersing a sample among a plurality of defined volumes;
(b) splitting said plurality of defined volumes, essentially simultaneously, into a plurality of matched pairs of daughter volumes comprising a plurality of first daughter volumes and a plurality of matched second daughter volumes, wherein said splitting is performed without the application of a pumping force to said defined volumes;
(c) conducting at least one analysis on said plurality of said first daughter volumes; and
(d) selecting a subset of said plurality of matched second daughter volumes based on said analysis.

37. The method of claim 36, wherein said analysis comprises a genetic assay or a functional assay.

38. (canceled)

39. The method of claim 36, wherein said sample comprises cells.

40.-41. (canceled)

42. The method of claim 36, wherein said sample comprises viruses.

43. The method of claim 36, wherein said sample comprises nucleic acids.

44. The method of claim 36, wherein said sample comprises multiple species of cells.

45. The method of claim 36, wherein said sample comprises antibiotics, chemotherapy agents, growth media, growth factors, or inhibitors.

46.-60. (canceled)

61. A method, comprising:
(a) providing a plurality of defined volumes, each of said plurality of defined volumes comprising one of a plurality of samples;
(b) subjecting said plurality of samples to a set of conditions;
(c) conducting a process on said plurality of samples;
(d) transferring said plurality of samples from said plurality of defined volumes to a shared container, thereby creating a pooled sample;
(e) conducting an analysis on said pooled sample; and
(f) determining from said analysis the extent to which said set of conditions enabled or did not enable said process.

62. The method of claim 61, wherein said set of conditions comprises the presence of an antibiotic or of a chemotherapy agent.

63. (canceled)

64. The method of claim 61, wherein said set of conditions comprises a given temperature or a given atmospheric condition.

65. (canceled)

66. The method of claim 61, wherein said set of conditions comprises the presence of a given organism, a growth factor, or an inhibitor.

67.-68. (canceled)

69. The method of claim 61, wherein said set of conditions comprises the absence of a nutrient.

70. The method of claim 61, wherein said process comprises cell growth.

71. The method of claim 61, wherein said process comprises nucleic acid amplification.

72. The method of claim 61, wherein said analysis comprises a genetic assay or a functional assay.

73.-77. (canceled)
